(19) United States
(12) Patent Application Publication
(10) Pub. No.: US 2024/0148659 A1
(43) Pub. Date: May 9, 2024

(54) LIQUID VISCOELASTIC SWALLOWING AID TO PROMOTE SAFE SWALLOWING OF SOLID ORAL DOSAGE FORMS (SODF)

(71) Applicant: SOCIETE DES PRODUITS NESTLE S.A., Vevey (CH)

(72) Inventors: ANAIS LAVOISIER, Vincennes (FR); MARCO RAMAIOLI, Gif sur Yvette (FR); MICHAEL REUBEN JEDWAB, Lausanne (CH); ADAM STEWART BURBIDGE, Arzier (CH); JAN ENGMANN, Lausanne (CH); SHREERAM SATHYAVAGEESWARAN, Singapore (SG)

(21) Appl. No.: 18/549,652

(22) PCT Filed: Mar. 11, 2022
(86) PCT No.: PCT/EP2022/056410

§ 371 Date: Sep. 8, 2023

(30) Foreign Application Priority Data
Mar. 12, 2021 (EP) 21162444.0

(51) Int. Cl. A61K 9/50; A23L 29/206; A61K 9/20

(52) U.S. Cl. CPC A61K 9/5036; A23L 29/206; A61K 9/205; A61K 9/5063

(57) ABSTRACT

The present disclosure is related to a liquid viscoelastic swallowing aid formulated to promote safe swallowing of Solid Oral Dosage Forms (SODF), e.g., tablets and/or capsules, in a patient in need thereof, and uses of such a liquid viscoelastic swallowing aid.

### FIELD OF INVENTION

The present disclosure is related to a liquid viscoelastic swallowing aid formulated to promote safe swallowing of Solid Oral Dosage Forms (SODF), e.g., tablets and/or capsules, in a healthy person or a patient in need thereof, and uses of such a liquid viscoelastic swallowing aid.

### BACKGROUND OF INVENTION

Solid Oral Dosage Forms (SODF), such as powders, granules, tablets and capsules are the most popular format for adult medications. Heppner et al., (2006) estimated that 65 to 70% of all medicines prescribed to patients in Germany in 2006 were tablets and capsules, intended to be swallowed as a whole. More recently, Schiele et al., (2013) reported that 90.1% of the drugs mentioned by patients attending general practices were tablets and capsules of different shapes and sizes.

Capsules and tablets remain the most popular oral drug delivery forms in the market because they are simple to handle, process, and store for industries and patients (Hoag 2017; Shaikh et al. 2018). However, it can be challenging to swallow them, which may lead to non-adherence to prescribed medicine. Medication-related swallowing difficulties affect between 10 and 60% of the adult population (Fields, Go, and Schulze 2015; Lau et al. 2015; Punzalan et al. 2019; Schiele et al. 2013; Strachan and Greener 2005; Tahaineh and Wazaify 2017), and have probably been underestimated in the past since people may be reluctant to seek advice from health professionals regarding such difficulties (Lau et al. 2015).

Patients may feel anxious about swallowing tablets and capsules because of anatomical features related to age and gender (dimensions and function of mouth, pharynx, upper esophageal sphincter and esophagus, etc.), physical characteristics of the dosage form itself (dimensions, surface properties, compliance, palatability, color, etc.) (Liu et al. 2016; Radhakrishnan 2016; Schiele et al. 2013; Shariff et al. 2020), or inappropriate swallowing techniques (Forough et al. 2018; Schiele et al. 2014).

Classical SODF are particularly troublesome for patients suffering from swallowing disorders (dysphagia), who are at higher risk for choking and silent aspiration (Schiele et al. 2015).

SODF may also stay trapped in the laryngeal folds and trigger local inflammations, esophagitis and ulcerations (U.S. Department of Health and Human Services Food and Drug Administration 2013). However, classical SODF can be also be troublesome for healthy persons and cause discomfort, such as an unpleasant feeling that the bolus sticks in the throat, etc.

Systematic in vivo studies about SODF swallowing are scarce and most of the data available in the literature focus on the effect of the tablet/capsule characteristics (e.g., size, shape, density, film coating) on the acceptability of the SODF. Kasashi et al., (2011) reported oral transit times between 0.95 and 1.45 s for large hard gelatin capsules (19 mm×7 mm) swallowed with water by healthy volunteers and evaluated with videofluoroscopy Yamamoto et al., (2014) showed that round biconvex tablets (up to 9 mm in diameter) affect swallowing behaviors in healthy persons. They reported an increase in the total number of swallows with increasing tablet size and number, as well as an increase in the EMG activity of the suprahyoid muscles (burst area and duration) when taking a round biconvex tablet (9 mm in diameter) compared to the water control. Schiele et al., (2015) showed that the addition of SODF to fluids or foods worsens the swallowing performances of stroke patients. They observed an increased risk in penetration and aspiration independently of the type and shape of the SODF.

Dysphagia is associated with various neurological, muscular, and respiratory disorders (strokes, Alzheimer's and Parkinson's diseases, metabolic myopathies, throat cancers, etc.), and with age-related physiological changes (Stegemann, Gosch, and Breitkreutz 2012). Given the current trend towards population ageing (United Nations 2020), dysphagia is a growing health concern which is believed to affect at least 15% of the elderly (Sura et al. 2012).

Furthermore, older adults are commonly prescribed multiple medications to manage multiple comorbidities (Masnoon et al. 2017), and most hypoglycemic agents, anti-hypertensives, or anti-dyslipidemia drugs are only available in SODF overlooking their special swallowing needs (Forough et al. 2018; Liu et al. 2016). Consequently, tablets and capsules are often manipulated by health care professionals or caregivers to facilitate their administration, but this has been related to an increased number of adverse events and medical errors (Logrippo et al. 2017; Nissen, Haywood, and Steadman 2009; Shariff et al. 2020).

Aside from drug compounding, other strategies may be used to help people struggling with tablets and capsules (Patel et al. 2020; Satyanarayana, Kulkarni, and Shivakumar 2011).

First, it may be possible to switch to another type of SODF (i.e., smaller in size, with a different shape or coating, chewable or orodispersible, etc.), to another pharmaceutical form (liquid or gel formulations, microparticles, etc.), or to a different route of administration (transdermal delivery for example). If this is not possible, swallowing assisting devices like cups and straws (Forough et al. 2018) and lubricant sprays (Diamond and Lavallee 2010) or coatings (Uloza, Uloziene, and Gradauskiene 2010) have been developed. Soft foods (puddings, apple sauce, yogurts, etc.) are also frequently used as swallowing-aid vehicles, but the compatibility between drug products and foods should be first carefully evaluated (Fukui 2015).

Recently, lubricant gels and thickened liquids specially designed to help swallowing whole SODF have appeared on the market (“Gloup”, “Slõ tablets”, “Medcoat”, or “Magic Jelly” for example). These products are inspired in products recommended for dysphagia management and are based on starch or gum-based viscoelastic materials. They are designed to increase swallowing comfort by masking the taste and transit of the SODF in the mouth and in the throat during swallowing. They also claim to support a smooth movement of the SODF from the mouth to the stomach by reducing the risk of adhesion (Fukui 2015). However, few studies have been published about those lubricant gels and they are only recommended for people without dysphagia at the moment (Malouh et al. 2020). Fukui et al., compared water to a swallowing aid (“Magic Jelly”, composed of agar, carrageenan, sugar, sugar alcohols, and flavors) used with placebo tablets and capsules (15 to 19 mm in diameter) by a group of 50 healthy people (20 to 50 years old). According to their sensory tests, the jelly was judged to be superior to water, useful, and safe, and their videofluoroscopic swallowing study (VFSS) revealed that capsules taken with the jelly took only 8 s to reach the stomach against 18 s for capsules swallowed with water (Fukui 2004, 2015). Wright et al., (2019) reported the results of a phase IV open-label randomized controlled cross-over trial (12 healthy males, aged 18-35 years), comparing aspirin tablets administered with water or encapsulated in a gelatin-based gel. The gel coating improved the taste and allowed the tablet to be swallowed without water, but the bioavailability of the drug was significantly reduced.

Regarding SODF swallowing for patients with dysphagia, Schiele et al., (2015) reported promising results from a video-endoscopic evaluation of 52 dysphagic stroke patients who swallowed medium-sized placebos with water thickened to pudding consistency, or milk: the prevalence rate of SODF swallowing difficulties was lower with texture-modified water than with milk, which suggests that tablets and capsules should rather be delivered with semisolids than fluids.

There is a general agreement that texture modification of liquids using shear thinning food thickeners promotes safe swallowing and helps managing dysphagia (Newman et al. 2016; Rofes et al. 2014), but the role of elastic and extensional properties of fluids on the dynamics of bolus transport has only recently been investigated and is still not fully understood (Hadde et al. 2019; Hadde, Chen, and Chen 2020; Mackley et al. 2013; Marconati and Ramaioli 2020; Nishinari et al. 2019; Sukkar et al. 2018). In a previous study, we observed that bolus elongation during in vitro swallowing and post-swallow residues were limited with thin elastic liquids (Marconati and Ramaioli 2020). A clinical study by Hadde et al., (2019) confirmed the effect of extensional properties on bolus elongation and safety, but no fluids with strong extensional properties were considered.

In view of the above the objective of this patent application and the accompanying study was to provide more efficient means to promote safe swallowing of Solid Oral Dosage Forms (SODFs) both in healthy individuals and in patients in need thereof, particularly in patients suffering from a swallowing disorder, such as patients suffering from dysphagia, or having various neurological, muscular, and respiratory disorders, such as strokes, Alzheimer's and Parkinson's diseases, metabolic myopathies, throat cancers, etc., or patients with age-related physiological changes (Stegemann, Gosch, and Breitkreutz 2012), or healthy individuals or patients that suffer from one of more diseases and require administration of SODF, such as older adults, typically having commonly prescribed multiple medications to manage multiple comorbidities, including e.g. hypoglycemic agents, anti-hypertensives, and/or anti-dyslipidemia drugs, etc.

### SUMMARY OF INVENTION

The underlying problem is particularly solved according to a first embodiment by a liquid viscoelastic swallowing aid for promoting safe swallowing of a Solid Oral Dosage Form (SODF), the liquid viscoelastic swallowing aid comprising a total amount from 0.1 to 10 wt % of a compound selected from beta-glucans, a plant-derived mucilage and/or a plant-extracted gum or a combination thereof, wherein the liquid viscoelastic swallowing aid comprises:
a shear viscosity from 10-1,000 mPa·s measured at a shear rate of 50 s−1 and 25° C.;
at least one extensional relaxation time as measured by a Capillary Breakup Extensional Rheometer (CaBER) at room temperature (25° C.) from 10 to 1,000 ms; and
optionally an IDDSI-level from 1 to 4, preferably measured at room temperature (25° C.).

As used herein, wt % refers to the weight of a particular feature per total weight.

The term “room temperature typically means 20-25° C., preferably 25° C. Where indicated in brackets, the temperature in the brackets is the preferred measurement temperature.

Preferably, the liquid viscoelastic swallowing aid according to the current invention, when measured in a Capillary Breakup Extensional Rheometer (CaBER) at 25° C., has a filament diameter that decreases exponentially in time.

In the liquid viscoelastic swallowing aid according to the current invention the Solid Oral Dosage Form (SODF) is preferably a tablet or a capsule. The capsule may have a standard size of between “5” to “000”. The tablet may have a length of 3 to 23 mm, preferably 3 to 22 mm, or may have a capsule length of 3 to 24 mm, preferably 3 to 22 mm.

The liquid viscoelastic swallowing aid according to the current invention may be in an administrable form, preferably selected from the group consisting of pharmaceutical formulations, dietary supplements, functional beverage products, food for special medical purpose (FSMP), and combinations thereof.

The liquid viscoelastic swallowing aid according to the invention may be in a concentrated form to be diluted prior to use or may be provided in a ready-to-use form. Alternatively, the liquid viscoelastic swallowing aid according to the invention may be provided as a powder to be reconstituted prior to use. However, all values as described herein for shear viscosity, extensional relaxation time and IDDSI-levels preferably refer to the reconstituted and hence final liquid viscoelastic swallowing aid ready for use.

Moreover, the current invention also concerns a liquid viscoelastic swallowing aid as described herein for use in promoting safe swallowing of a Solid Oral Dosage Form (SODF) either in a patient in need of such a treatment or in a healthy person.

Hence, according to a second embodiment, the invention concerns a liquid viscoelastic swallowing aid as described herein for use in promoting safe swallowing of a Solid Oral Dosage Form (SODF) in a patient in need thereof. Such a patient in need thereof may be a patient suffering from a swallowing disorder, from dysphagia or from a compromised secretion of saliva, or may have an increased risk for choking and silent aspiration, or may be a patient being prescribed multiple medications, e.g. a child or an older patient suffering from multiple comorbidities and being prescribed multiple medications, or being a cancer patient, or suffering from hyperglycemia, diabetes, cardiovascular disease, arthritis, hypertension, asthma, dementia, MCI, Alzheimer's disease, Parkinson's disease, epilepsy, motor neuron disease, amyotrophic lateral sclerosis (ALS), multiple sclerosis (MS), osteoporosis, stroke, chronic kidney disease, or deep vein thrombosis, etc.

Moreover, according to a third embodiment, the invention concerns a non-therapeutic use of a liquid viscoelastic swallowing aid as described herein for promoting safe swallowing of a Solid Oral Dosage Form (SODF) in a healthy person. Generally, such a healthy person is typically a person that does not suffer from any of such diseases as described herein. Such a healthy person may prefer using a liquid viscoelastic swallowing aid as described herein to avoid an uncomfortable experience when swallowing such an SODF. SODF in this context may be e.g., common nutritional supplements, vitamins, but also common medicaments, such as tablets against pain, headache, migraine, menstrual pain, back pain, etc.

Furthermore, according to a fourth embodiment the invention is also directed to a method of administering or feeding the liquid viscoelastic swallowing aid as described herein to a patient in need thereof for promoting swallowing of a SODF.

Any of the herein defined preferred features, embodiments or alternatives may be combined in a suitable manner if not otherwise disclosed.

For a complete understanding of the present invention and the advantages thereof, reference is made to the following detailed description.

### DETAILED DESCRIPTION

The inventors of the current patent application have surprisingly found that the underlying problem can be efficiently solved by a liquid viscoelastic swallowing aid as described herein, suitable to promote safe swallowing of Solid Oral Dosage Forms (SODF) either in a healthy person or in a patient in need thereof. The current patent application is particularly based on the novel and surprising finding of the inventors using an in vitro model for swallowing that the rheological dynamics of specific viscoelastic liquid carriers provides a more efficient process of swallowing SODF during the oral phase. The use of such specific viscoelastic liquid carriers also leads to a lowered risk of bolus extension or bolus rupturing during the swallowing process and hence also to a significantly lower risk of aspiration of the bolus or parts thereof during the swallowing process. Moreover, the use of such specific viscoelastic liquid carriers also enables in a particularly preferable manner the non-therapeutic administration of SODF.

As used herein the feature “bolus” includes any entity of the liquid viscoelastic swallowing aid and the SODF formed in the mouth in preparation for swallowing. The bolus may be of any size, composition and/or texture.

The rheological properties of the inventive liquid viscoelastic swallowing aid comprising beta-glucans, a plant-derived mucilage and/or a plant-extracted gum or a combination thereof as a carrier is characterized by a certain shear and extensional viscosity as well as a specific flow behavior, characterized by IDDSI level and shear viscosity. These factors together result in an unexpected and surprising effect on bolus velocity, bolus shape, post-swallow residues and SODF position in the bolus. Particularly the latter has been surprisingly identified as a novel and powerful variable to discriminate the effectiveness of different carriers.

Generally, inventors found that when swallowed with water, capsules and tablets did not impact significantly the velocity of the bolus, but they lagged behind the liquid bolus, suggesting that low viscosity Newtonian fluids are not efficient carriers for SODF. Increasing the viscosity of the investigated carriers at high shear rates (i.e., ≥300 s−1) improved the ability of the liquid to transport the SODF but also increased the amount of post-swallow residues. At equivalent shear viscosity, the elastic and extensional properties of the carriers influenced positively the position of the SODF in the bolus. Capsules and tablets were transported toward the front of these boluses, during the oral phase of swallowing, which is considered positive to avoid SODF sticking to the mucosae in the following stages of swallowing. As a surprising result thin elastic liquids were thus identified under specific conditions to better promote safe swallowing of capsules and tablets.

The surprising finding of the inventors was evidenced using an in vitro model for swallowing, thereby evaluating the effect of the rheology of different liquid carriers on the oral phase of swallowing of capsules and tablets in vitro, and in particular whether the transport of SODF from the oral cavity to the pharynx is facilitated by the use of elastic liquids. The rheological properties of a selection of liquid carriers were characterized using shear and extensional rheometry, and in vitro swallowing experiments were performed with a capsule or a tablet in order to explore the swallowing dynamics of different combinations of carrier and SODF.

According to a first embodiment, the current invention therefore provides a liquid viscoelastic swallowing aid for promoting safe swallowing of a Solid Oral Dosage Form (SODF). The inventive liquid viscoelastic swallowing aid preferably comprises a total amount from 0.1 to 10 wt % of a compound selected from beta-glucans, a plant-derived mucilage and/or a plant-extracted gum or a combination thereof.

Moreover, the inventive liquid viscoelastic swallowing aid preferably comprises:
a shear viscosity from 10-1,000 mPa·s measured at a shear rate of 50 s−1 and 25° C.;
at least one extensional relaxation time as measured by a Capillary Breakup Extensional Rheometer (CaBER) at 25° C. from 10 to 1,000 ms; and
optionally an IDDSI-level from 1 to 4, preferably measured at room temperature (25° C.).

Typically, the beta-glucans, plant-extracted gums and/or plant-derived mucilages, or a combination thereof, are present in the liquid viscoelastic swallowing aid in a total amount from 0.01 wt % to 10 wt %, preferably from 0.1 wt % to 7.5 wt %, and most preferably from 0.1 wt % to 5 wt %, e.g., from 0.1 wt % to 5 wt %, from 0.1 wt % to 4.5 wt %, from 0.1 wt % to 3.5 wt %. The lower limits of such ranges may be increased from 0.1 wt % to e.g., 0.25 wt %, 0.5 wt %, 0.75 wt % or 1.0 wt %. Any of such lower limits may be combined with the above-mentioned ranges.

In the present invention, beta-glucans (β-glucans) typically refer to homopolysaccharides of D-glucopyranose monomers linked by (1→3), (1→4)-β-glycosidic bonds. Beta-glucans are derivable from plant or microbial origin, typically as a cereal extract, e.g., from oat or barley, by methods known to the skilled person, for example as described by Lazaridou et al. in ‘A comparative study on structure-function relations of mixed-linkage (1→3), (1→4) linear β-D-glucans' in Food Hydrocolloids, 18 (2004), 837-855.

Beta-glucans and hence also oat shows particularly preferable properties in the inventive liquid viscoelastic swallowing aid as it allows to provide with small amounts of beta-glucans the claimed shear viscosities of 10 to 1,000 mPa·s when measured at a shear rate of 50 s−1 at 25° C.

Similarly, plant-extracted gums and/or plant-derived mucilages provide superior properties, e.g., over xanthan, when used herein.

Thereby, the plant-extracted gums in the liquid viscoelastic swallowing aid are preferably selected from the group consisting of plant-extracted gums, plant-derived mucilages and combinations thereof. The plant-extracted gums may further be selected from the group consisting of okra gum, konjac mannan, tara gum, locust bean gum, guar gum, fenugreek gum, tamarind gum, cassia gum, acacia gum, gum ghatti, pectins, cellulosics, tragacanth gum, karaya gum, or any combinations thereof. In a preferred embodiment, the plant-extracted gum is okra gum. Extraction of plant-extracted gums may be carried out by procedures known to a skilled person.

Moreover, the plant-derived mucilages for the liquid viscoelastic swallowing aid are selected from the group consisting of cactus mucilage (Ficus indica), psyllium mucilage (Plantago ovata), mallow mucilage (Malva sylvestris), flax seed mucilage (Linum usitatissimum), marshmallow mucilage (Althaea officinalis), ribwort mucilage (Plantago lanceolata), mullein mucilage (Verbascum), cetraria mucilage (Lichen islandicus), or any combinations thereof. In a preferred embodiment, the plant-derived mucilage is cactus mucilage (Ficus indica). Extraction of plant-derived mucilages may be carried out by procedures known to a skilled person.

It is particularly preferred that the food grade polymer is selected from okra gum and/or cactus mucilage (Ficus indica), or a combination thereof.

It is even more preferred that the liquid viscoelastic swallowing aid according to the invention contains a compound selected from the group comprising or consisting of beta-glucans, a plant-derived mucilage and/or a plant-extracted gum or a combination thereof, preferably a compound selected from the group comprising or consisting of beta-glucans, okra gum and/or cactus mucilage (Ficus indica), or a combination thereof. Amounts are thereby from 0.1 to 10 wt % as defined herein above.

In a further preferred embodiment, the liquid viscoelastic swallowing aid according to the invention does not contain starch, such as waxy maize starch, or xanthan gum, modified xanthan gum such as non-pyruvylated xanthan gum or reduced-pyruvylated xanthan gum, carageenan, or a combination thereof. Preferably, it does not contain a combination of starch and carrageenan or a combination of casein and waxy maize starch.

Furthermore, the liquid viscoelastic swallowing aid according to the first embodiment of the invention optionally has an IDDSI level from 1 to 4, preferably from 1 to 3, inter alia due to residual shear viscosity. In this context, IDDSI levels and methods how to determine IDDSI levels are well known to a skilled person. Quite recently, the IDDSI system has appeared as an approach to characterize the flow properties of liquids, particularly Texture-Modified Foods and Thickened Fluids Used in Dysphagia Management. The IDDSI system is mainly based on volume emptying of a calibrated syringe. The levels for liquids/drinks characterized in the IDDSI measurement are defined as levels 0-4, also termed level 0=“thin”, 1=“slightly thick”, level 2=“mildly thick”, level 3=“moderately thick”, and level 4=“extremely thick”. Measurement of such levels is preferably carried out as defined by the International Dysphagia Diet Standardisation Initiative (IDDSI), e.g., available under https://link.springer.com/article/10.1007/s00455-016-9758-y, or as published in Cichero, J. A. Y., Lam, P., Steele, C. M. et al. Development of International Terminology and Definitions for Texture-Modified Foods and Thickened Fluids Used in Dysphagia Management: The IDDSI Framework. Dysphagia 32, 293-314 (2017). https://doi.org/10.1007/s00455-016-9758-y.

In view of the above, the current application therefore defines rheological properties of the inventive liquid viscoelastic swallowing aid optionally also on basis of IDDSI levels 1, 2, 3 and 4, corresponding to stages 1=“thin”, 2=“mildly thick”, 3=“moderately thick” and 4=“extremely thick”. Such rheological properties, or also flow behavior in case of liquids (as well as the shear viscosity values and extensional relaxation times), always refer to the (final) liquid viscoelastic swallowing aid, i.e., the liquid viscoelastic swallowing aid ready for use in swallowing a Solid Oral Dosage Form (SODF) by a healthy person or a patient in need thereof according to the current invention.

In the context of the current invention the IDDSI flow test is preferably run at room temperature (typically 20-25° C., preferably 25° C.) in triplicate to evaluate the IDDSI level of each liquid carrier (IDDSI 2019). In this test, a standard luer slip tip syringe is filled up to the 10 mL mark with the sample, and the liquid is then allowed to flow for 10 s. Based on the remaining volume left in the syringe, liquid samples are categorized in four levels of increasing thickness: Level 0 (less than 1 mL remaining), Level 1 (1-4 mL remaining), Level 2 (4-8 mL remaining), Level 3 (not less than 8 mL remaining). If the liquid does not flow through the tip of the syringe, it is classified as Level 4. IDDSI Level 4 liquids can also be evaluated with the IDDSI spoon tilt test: they must hold their shape on a spoon and fall off easily if the spoon is tilted. Reference is made to the above-described measurement methods for further details.

Optionally, the liquid viscoelastic swallowing aid according to the first embodiment of the invention has an IDDSI level from 1 to 4, more preferably from 1 to 3, likewise preferably from 1 to 2 or from 2 to 3, more preferably from 1 to 2, even more preferably from 1 to 2, e.g., 1 or 2, most preferably 1, preferably measured at room temperature (25° C.).

Furthermore, the liquid viscoelastic swallowing aid according to the invention typically comprises a shear viscosity from 10 to 1,000 mPa·s, a shear viscosity from 10 to 900 mPa·s, a shear viscosity from 10 to 800 mPa·s, or even a shear viscosity from 10 to 700 mPa·s, each measured at a shear rate of 50 s−1 and 25° C. Likewise, the liquid viscoelastic swallowing aid according to the invention comprises a shear viscosity from 10 to 600 mPa·s measured at a shear rate of 50 s−1 and 25° C., preferably a shear viscosity from 10 to 500 mPa·s measured at a shear rate of 50 s−1 and 25° C., likewise preferably a shear viscosity from 10 to 400 mPa·s measured at a shear rate of 50 s−1 and 25° C., more preferably a shear viscosity from 10 to 350 mPa·s, measured at a shear rate of 50 s−1 and 25° C., e.g. a shear viscosity from 10 to 350 mPa·s, measured at a shear rate of 50 s−1 and 25° C., a shear viscosity from 20 to 350 mPa·s, measured at a shear rate of 50 s−1 and 25° C., or even a shear viscosity from 30 to 350 mPa·s, measured at a shear rate of 50 s−1 and 25° C. Such values particularly include a shear viscosity from 10 to 300 mPa·s measured at a shear rate of 50 s−1 and 25° C., from 10 to 200 mPa·s measured at a shear rate of 50 s−1 and 25° C., even more preferably a shear viscosity from 10 to 100 mPa·s measured at a shear rate of 50 s−1 and 25° C., and most preferably a shear viscosity from 10 to 50 mPa·s measured at a shear rate of 50 s−1 and 25° C., or from 10 to 40 mPa·s or even from 10 to 30 mPa·s, each of the above defined shear viscosity levels measured at a shear rate of 50 s−1 and 25° C.

In the context of the current invention the shear rate is preferably measured with a Modular Compact Rheometer (MCR) 102 (Anton Paar GmbH, Graz, Austria), at 25° C. A cone and plate geometry (diameter=50 mm, cone angle=4°, truncation=500 μm), and a 0.5 mm gap used to obtain flow curves in a range of shear rates between 0.5 and 800 reciprocal seconds. Three repetitions are preferably performed for each sample.

The liquid viscoelastic swallowing aid according to the invention comprises at least one extensional relaxation time as measured by a Capillary Breakup Extensional Rheometer (CaBER). As used herein the Capillary Breakup Extensional Rheometer (CaBER) is a device suitable to measure complex fluids that contain strong extensional flow fields in liquid samples. Preferably, the Capillary Breakup Extensional Rheometer (CaBER) as used herein is a HAAKE CaBER 1 from Thermo Fisher Scientific, the currently only commercially available Extensional Rheometer for fluids on the market. The measurement of extensional relaxation times as determined in the context of the current invention is therefore preferably carried out using as a Capillary Breakup Extensional Rheometer (CaBER) a Thermo Scientific HAAKE CaBER 1 from Thermo Fisher Scientific, under ambient conditions as defined in the instruction manual HAAKE CaBER 1, version 1.8, from Thermo Fisher Scientific, p. 19, section 8.4. “Ambient conditions according to EN 61010”, namely in an air-conditioned room, at ambient temperatures, between 20 and 25° C., preferably 25° C., indoors, maximally 2000 meters above sea level.

During the CaBER experiment as performed herein for measuring the relaxation time of the inventive liquid viscoelastic swallowing aid, a drop of said product is preferably placed between two vertically aligned and parallel circular metal surfaces, both having a diameter of 6 mm. The metal surfaces are then rapidly separated linearly over a time interval of 50 ms (milliseconds). The filament formed by this stretching action subsequently thins under the action of interfacial tension and the thinning process is followed quantitatively using a digital camera and/or laser sheet measuring the filament diameter at its mid-point. The relaxation time in a CaBER experiment is determined by plotting the normalised natural logarithm of the filament diameter during the thinning process versus time and determining the slope of the linear portion (dₗₙ (D/Dₒ)/dₜ) of this curve, where D is the filament diameter, Dₒ the filament diameter at time zero and t the time of filament thinning. The relaxation time in this context is then defined as

−1/(3dₗₙ(D/Dₒ)/dₜ).

The HAAKE CaBER 1 may be adapted for measurement such that the initial separation between the two circular plates (6 mm in diameter) is set at 3 mm, and an axial displacement up to 10 mm is imposed in 50 ms to drive the filament thinning. The evolution in time of the midpoint diameter of the thread can be measured with a laser micrometer with a beam thickness of 1 mm and a resolution of 20 μm. The extensional relaxation time can be calculated with the CaBER Analysis software (Haake RheoWin Software, version 5.0.12) by fitting the data with the elastic (exponential) model. Moreover, high-speed videos of the experiments can be taken at 1,000 frames per second to record the shape evolution of the capillary thread using a Phantom V1612 high-speed camera (Vision Research, Wayne, NJ), to monitor the shape-evolution of the sample.

Preferably, the filament diameter of the liquid viscoelastic swallowing aid as measured by a Capillary Breakup Extensional Rheometer (CaBER) at room temperature (25° C.) for determining the extensional relaxation time as described herein decreases exponentially in time during the CaBER experiment.

Likewise, the liquid viscoelastic swallowing aid according to the invention comprises at least one extensional relaxation time as measured by a Capillary Breakup Extensional Rheometer (CaBER) at room temperature (25° C.) from 10 to 1,000 ms. Preferably, the liquid viscoelastic swallowing aid according to the invention comprises at least one extensional relaxation time as measured by a Capillary Breakup Extensional Rheometer (CaBER) at room temperature (25° C.) from 10 to 900 ms, likewise preferably from 10 to 800 ms, more preferably from 10 to 700 ms, likewise more preferably from 10 to 600 ms, even more preferably from 10 to 500 ms, such as from 10 to 475 ms, preferably from 10 ms to 450 ms, likewise preferably, more preferably from 10 ms to 425 ms, likewise more preferably from 10 ms to 400 ms, even more preferably from 10 ms to 375 ms, and most preferably from 10 ms to 350 ms, including values from 10 ms to 350 ms, from 20 ms to 350 ms, from 30 ms to 350 ms, from 40 ms to 350 ms, from 50 ms to 350 ms, from 60 ms to 350 ms, from 70 ms to 350 ms, from 80 ms to 350 ms, from 90 ms to 350 ms, from 100 ms to 350 ms, and also including values from 10 ms to 150 ms, from 20 ms to 150 ms, from 30 ms to 150 ms, from 40 ms to 150 ms, from 50 ms to 150 ms, from 60 ms to 150 ms, from 70 ms to 150 ms, from 80 ms to 150 ms, from 90 ms to 150 ms, from 100 ms to 150 ms, each extensional relaxation time measured by a Capillary Breakup Extensional Rheometer (CaBER) at room temperature (25° C.). All combinations of ranges are included in the liquid viscoelastic swallowing aid according to the invention.

According to a preferred embodiment, the liquid viscoelastic swallowing aid according to the invention comprises a total amount from 0.01 wt % to 10 wt %, preferably from 0.1 wt % to 7.5 wt %, of a compound selected from beta-glucans, a plant-derived mucilage and/or a plant-extracted gum or a combination thereof and preferably comprises:
a shear viscosity from 10-1,000 mPa·s measured at a shear rate of 50 s−1 and 25° C., preferably a shear viscosity from 10 to 900 mPa·s, measured at a shear rate of 50 s−1 and 25° C., more preferably a shear viscosity from 10 to 800 mPa·s, or below as defined above, all measured at a shear rate of 50 s−1 and 25° C.;
at least one extensional relaxation time as measured by a Capillary Breakup Extensional Rheometer (CaBER) at room temperature from 10 to 1,000 ms, preferably from 10 to 800 ms, more preferably from 10 ms to 600 ms, or below as defined above each extensional relaxation time measured by a Capillary Breakup Extensional Rheometer (CaBER) at room temperature (25° C.); and
optionally an IDDSI-level from 1 to 4, preferably from 1 to 3 or from 1 to 2, preferably measured at room temperature (25° C.).

According to a particularly preferred embodiment, the liquid viscoelastic swallowing aid according to the invention comprises a total amount from 0.01 wt % to 10 wt %, preferably from 0.1 wt % to 7.5 wt %, more preferably from 0.1 wt % to 5 wt %, e.g., from 0.1 wt % to 5 wt %, from 0.1 wt % to 4.5 wt %, from 0.1 wt % to 3.5 wt % of a compound selected from beta-glucans, a plant-derived mucilage and/or a plant-extracted gum or a combination thereof, and preferably comprises:
a shear viscosity from 10 to 800 mPa·s measured at a shear rate of 50 s−1 and 25° C., preferably a shear viscosity from 10 to 700 mPa·s measured at a shear rate of 50 s−1 and 25° C., more preferably a shear viscosity from 10 to 600 mPa·s, all measured at a shear rate of 50 s−1 and 25° C.;
at least one extensional relaxation time as measured by a Capillary Breakup Extensional Rheometer (CaBER) at room temperature (25° C.) from 10 to 600 ms, preferably from 10 ms to 500 ms, likewise preferably, more preferably from 10 ms to 400 ms; and
optionally an IDDSI-level from 1 to 4, preferably 1 to 3, more preferably from 1 to 2, preferably 1, preferably measured at room temperature (25° C.).

According to a likewise preferred embodiment, the liquid viscoelastic swallowing aid according to the invention comprises a total amount from 0.1 wt % to 5 wt %, preferably from 0.1 wt % to 4.5 wt %, e.g., from 0.1 wt % to 5 wt %, from 0.1 wt % to 4.5 wt %, from 0.1 wt % to 3.5 wt % of a compound selected from beta-glucans, a plant-derived mucilage and/or a plant-extracted gum or a combination thereof, and preferably comprises:
a shear viscosity from 10 to 600 mPa·s measured at a shear rate of 50 s−1 and 25° C., preferably a shear viscosity from 10 to 500 mPa·s measured at a shear rate of 50 s−1 and 25° C., more preferably a shear viscosity from 10 to 400 mPa·s, all measured at a shear rate of 50 s−1 and 25° C.;
at least one extensional relaxation time as measured by a Capillary Breakup Extensional Rheometer (CaBER) at room temperature (25° C.) from 10 to 400 ms, preferably from 10 ms to 375 ms, more preferably from 10 ms to 350 ms; and
optionally an IDDSI-level from 1 to 4, preferably 1 to 3, more preferably from 1 to 2, preferably 1, preferably measured at room temperature (25° C.).

According to an even more preferred embodiment, the liquid viscoelastic swallowing aid according to the invention comprises a total amount from 0.1 wt % to 4.5 wt %, preferably from 0.1 wt % to 3.5 wt %, of a compound selected from beta-glucans, a plant-derived mucilage and/or a plant-extracted gum or a combination thereof, and preferably comprises:
a shear viscosity from 10 to 400 mPa·s measured at a shear rate of 50 s−1 and 25° C., preferably a shear viscosity from 10 to 300 mPa·s measured at a shear rate of 50 s−1 and 25° C., more preferably a shear viscosity from 10 to 200 mPa·s, all measured at a shear rate of 50 s−1 and 25° C.;
at least one extensional relaxation time as measured by a Capillary Breakup Extensional Rheometer (CaBER) at room temperature (25° C.) from 10 ms to 150 ms or from 20 ms to 150 ms, preferably from 30 ms to 150 ms or from 40 ms to 150 ms, more preferably from 50 ms to 150 ms, likewise more preferably from 60 ms to 150 ms, even more preferably from 70 ms to 150 ms, and most preferably from 80 ms to 150 ms, e.g. from 90 ms to 150 ms, or from 100 ms to 150 ms; and
optionally an IDDSI-level from 1 to 4, preferably 1 to 3, more preferably from 1 to 2, preferably 1, preferably measured at room temperature (25° C.).

Compounds for the inventive liquid viscoelastic swallowing aid can be selected from food grade polymers beta-glucans, plant-extracted gums and/or plant-derived mucilages, or a combination thereof as defined above.

The liquid viscoelastic swallowing aid according to the invention generally may be provided in a ready-to-use form, or may be provided in a concentrated liquid form, such as a highly viscous composition or a gel or gel like composition, to be reconstituted prior to use, e.g., diluted with water, or may be provided as a powder to be reconstituted prior to use.

Alternatively, the liquid viscoelastic swallowing aid may be provided in dry form, such as a powder, wherein, upon adding an appropriate amount of water, the nutritional product as defined herein can be reconstituted to exhibit the properties of the liquid viscoelastic swallowing aid as claimed and described herein. Reconstitution herein typically means the addition of an appropriate amount of water to any of the concentrated or dry forms of the liquid viscoelastic swallowing aid, e.g., as a concentrate, a gel, a powder, etc., to arrive at the herein defined (final) concentrations of a ready-to-use product as claimed.

In the current invention, the IDDSI values, the shear viscosity data and the extensional viscosity data as defined herein for the inventive liquid viscoelastic swallowing aid therefore always concern the ready-to-use liquid composition or a reconstituted liquid composition ready to be used as a liquid viscoelastic swallowing aid. In other words, the inventive liquid viscoelastic swallowing aid, when provided e.g., in a more concentrated or dry form or a powder form, is to be reconstituted and then forms the inventive liquid viscoelastic swallowing aid with IDDSI values, shear viscosity data and extensional viscosity data as defined herein for the inventive liquid viscoelastic swallowing aid.

The liquid viscoelastic swallowing aid may be provided in a sachet, a bottle, a dispensing device such as a metered pump dispenser, as a single use pack, mini Gualapack, water-soluble packs, etc.

The inventive liquid viscoelastic swallowing aid may be provided in an amount between 5 and 200 ml, preferably between 5 and 150 ml, more preferably between 5 and 100 ml, e.g., between 10 and 100 ml, between 20 and 100 ml, between 30 and 100 ml, between 40 and 100 ml, between 50 and 100 ml, between 10 and 90 ml, between 10 and 80 ml, between 10 and 70 ml, between 10 and 60 ml, between 10 and 50 ml, etc. Such amounts are preferably single-dosage amounts for swallowing one or more of a Solid Oral Dosage Form (SODF) by a patient in need thereof. Any of the above-mentioned sachet, bottle, or dispensing device, etc. may provide such an amount for a single application. Alternatively, larger volumes may be provided for multiple applications, e.g., between 50 and 1,000 ml, or even more, e.g., in a bottle of the corresponding size.

The liquid viscoelastic swallowing aid according to the invention is preferably provided in an administrable form selected from the group consisting of pharmaceutical formulations, dietary supplements, functional beverage products, food for special medical purpose (FSMP), and combinations thereof.

The inventive liquid viscoelastic swallowing aid is preferably stable for at least several months (1, 2, 3, 4, 5, 6), preferably at least six months and more preferably at least 1 year, usually at room temperature, preferably when stored, e.g., in a closed bottle or closed container typically filled under aseptic conditions, or cold filled, e.g., in the presence of preservatives. Stable is interpreted as meaning that the viscosity and the claimed rheological properties remain about constant during envisaged shelf-life time. Preferably, stable is also interpreted such that the microbial count remains about constant during envisaged shelf-life time. Accordingly, the inventive liquid viscoelastic swallowing aid can be provided as a pre-packaged product, such as sachet, a bottle, a dispensing device such as a metered pump dispenser, to the end user as described below. The end user can use an adequate amount of the inventive liquid viscoelastic swallowing aid, e.g., to be easily dispensed or squeezed out from a bottle or sachet, to be mixed with the SODF to allow safe swallowing of the SODF and the formed bolus. The bolus is as defined above, i.e., any mixture of the SODF and the inventive liquid viscoelastic swallowing aid The inventive liquid viscoelastic swallowing aid is moreover understood to include any number of optional ingredients. In case, such optional ingredients are contained, the inventive liquid viscoelastic swallowing aid nevertheless has to provide the IDDSI levels, shear viscosity and extensional relaxation times as defined above. A skilled person will therefore add such further ingredients only in amounts that still allow achieving the herein defined values for IDDSI levels, shear viscosity and extensional relaxation times as defined above. Nevertheless, there may be also an inventive liquid viscoelastic swallowing aid containing at least one or more of such optional further ingredients.

The optional ingredients may include conventional food additives, for example one or more, acidulants, additional thickeners, buffers or agents for pH adjustment, chelating agents, colorants, emulsifiers, excipient, flavour agent, minerals, osmotic agents, a pharmaceutically acceptable carrier, preservatives, stabilisers, sugar(s), sweetener(s), texturiser(s), pharmaceutical ingredients, immunity boosting ingredients, prebiotics, antioxidants, salts, e.g. salts for rehydration, and/or vitamin(s), etc. Such optional ingredients may in addition also contain proteins, lipids and carbohydrates, as defined below.

The optional ingredients can be added in any suitable amount, provided the herein defined values for IDDSI levels, shear viscosity and extensional relaxation times of the inventive liquid viscoelastic swallowing aid remain as defined above.

The inventive liquid viscoelastic swallowing aid may therefore optionally comprise at least one protein. The at least one protein can be a dairy based protein, a plant-based protein or an animal-based protein or any combination thereof. Dairy-based proteins include, for example, casein, caseinates (e.g., all forms including sodium, calcium, potassium caseinates), casein hydrolysates, whey (e.g., all forms including concentrate, isolate, demineralized), whey hydrolysates, milk protein concentrate, and milk protein isolate. Plant-based proteins include, for example, soy protein (e.g., all forms including concentrate and isolate), pea protein (e.g., all forms including concentrate and isolate), canola protein (e.g., all forms including concentrate and isolate), other plant proteins that commercially are wheat and fractionated wheat proteins, corn and it fractions including zein, rice, oat, potato, peanut, green pea powder, green bean powder, and any proteins derived from beans, lentils, and pulses. Animal-based proteins may be selected from the group consisting of beef, poultry, fish, lamb, seafood, or combinations thereof.

In a further aspect of the first embodiment of the present invention, the inventive liquid viscoelastic swallowing aid may optionally comprise a source of fat. The source of fat includes, vegetable fat (such as olive oil, corn oil, sunflower oil, rapeseed oil, hazelnut oil, soy oil, palm oil, coconut oil, canola oil, lecithins, and the like), animal fats (such as milk fat) or any combinations thereof.

In another aspect of the first embodiment of the present invention, the inventive liquid viscoelastic swallowing aid may optionally comprise fibres or a fibre blend. The fibre blend may contain a mixture of soluble and insoluble fibres. Soluble fibres may include, for example, fructooligosaccharides, acacia gum, inulin, etc. Insoluble fibres may include, for example, pea outer fibre.

In a further aspect of the first embodiment of the present invention, the inventive liquid viscoelastic swallowing aid may optionally comprise a source of carbohydrate. The source of carbohydrate includes sucrose, lactose, glucose, fructose, corn syrup solids, maltodextrin, modified starch, amylose starch, tapioca starch, corn starch or any combinations thereof.

Inclusion of carbohydrates are advantageous inter alia to allow a simple preparation of the nutritional product, e.g., in form of a dispersion of a powder, etc.

In one other aspect of the first embodiment, the inventive liquid viscoelastic swallowing aid may optionally comprise at least one the following prebiotics, or any combination thereof: fructooligosaccharides, fucosyllactose, galactooligosaccharides, galactomannans, gentiooligosaccharides, glucooligosaccharides, guar gum, inulin, isomalto-oligosaccharides, lactoneotetraose, lactosucrose, lactulose, levan, maltodextrins, milk oligosaccharides, partially hydrolyzed guar gum, pecticoligosaccharides, resistant starches, retrograded starch, sialooligosaccharides, sialyllactose, soyoligosaccharides, sugar alcohols, xylooligosaccharides, or their hydrolysates, or combinations thereof. The prebiotic is a food substance that selectively promotes the growth of beneficial bacteria or inhibits the growth or mucosal adhesion of pathogenic bacteria in the intestines. The prebiotics are not inactivated in the stomach and/or upper intestine or absorbed in the gastrointestinal tract of the individual ingesting them, but they are fermented by the gastrointestinal microflora and/or by probiotics. Prebiotics are, for example, defined by Glenn R. Gibson and Marcel B. Roberfroid, Dietary Modulation of the Human Colonic Microbiota: Introducing the Concept of Prebiotics, J. Nutr. 1995 125: 1401-1412. fff

In a further aspect of the first embodiment, the inventive liquid viscoelastic swallowing aid may optionally comprise at least one probiotic. Probiotics are food-grade microorganisms (alive, including semi-viable or weakened, and/or non-replicating), metabolites, microbial cell preparations or components of microbial cells that could confer health benefits on a host when administered, more specifically probiotics beneficially affect the host by improving intestinal microbial balance, leading to effects on the health or well-being of the host. See, Salminen S, Ouwehand A. Benno Y. et al., Probiotics: how should they be defined? Trends Food Sci. Technol. 1999:10, 107-10. In general, it is believed that these probiotics inhibit or influence the growth and/or metabolism of pathogenic bacteria in the intestinal tract. The probiotics may also activate the immune function of the host. The probiotics used in the present invention include Aerococcus, Aspergillus, Bacillus, Bacteroides, Bifidobacterium, Candida, Clostridium, Debaromyces, Enterococcus, Fusobacterium, Lactobacillus, Lactococcus, Leuconostoc, Melissococcus, Micrococcus, Mucor, Oenococcus, Pediococcus, Penicillium, Peptostrepococcus, Pichia, Propionibacterium, Pseudocatenulatum, Rhizopus, Saccharomyces, Staphylococcus, Streptococcus, Torulopsis, Weissella, or any combination thereof.

The inventive liquid viscoelastic swallowing aid may optionally also comprise a synbiotic in one aspect of the first embodiment. A synbiotic is a supplement that comprises both a prebiotic (at least one of the aforementioned) and a probiotic (at least one of the aforementioned) that work together to improve the microflora of the intestine.

In a further aspect of the first embodiment, the inventive liquid viscoelastic swallowing aid may optionally comprise at least one the following amino acids, or any combination thereof: alanine, arginine, asparagine, aspartate, citrulline, cysteine, glutamate, glutamine, glycine, histidine, hydroxyproline, hydroxyserine, hydroxytyrosine, hydroxylysine, isoleucine, leucine, lysine, methionine, phenylalanine, proline, serine, taurine, threonine, tryptophan, tyrosine and valine.

In a further aspect of the first embodiment, the inventive liquid viscoelastic swallowing aid may optionally comprise at least one fatty acid or any combination thereof. The fatty acid includes ω-3 fatty acids such α-linolenic acid (“ALA”), docosahexaenoic acid (“DHA”) and eicosapentaenoic acid (“EPA”). The fatty acid is derivable from fish oil, krill, poultry, eggs, a plant source, algae and a nut source. The nut source includes flax seed, walnuts, almonds.

In a further aspect of the first embodiment, the inventive liquid viscoelastic swallowing aid may optionally comprise at least one phytonutrient. The phytonutrient is at least one of flavanoids, allied phenolic compounds, polyphenolic compounds, terpenoids, alkaloids, sulphur-containing compounds.

Phytonutrient are non-nutritive compounds that are found in many foods. Phytonutrients are functional foods that have health benefits beyond basic nutrition, and are health promoting compounds that come from plant sources. Phytonutrient refers to any chemical produced by a plant that imparts one or more health benefit on a user. Non-limiting examples of phytonutrients include those that are:
i) phenolic compounds which include monophenols (such as, for example, apiole, carnosol, carvacrol, dillapiole, rosemarinol); flavonoids (polyphenols) including flavonols (such as, for example, quercetin, fingerol, kaempferol, myricetin, rutin, isorhamnetin), flavanones (such as, for example, fesperidin, naringenin, silybin, eriodictyol), flavones (such as, for example, apigenin, tangeritin, luteolin), flavan-3-ols (such as, for example, catechins, (+)-catechin, (+)-gallocatechin, (−)-epicatechin, (−)-epigallocatechin, (−)-epigallocatechin gallate (EGCG), (−)-epicatechin 3-gallate, theaflavin, theaflavin-3-gallate, theaflavin-3′-gallate, theaflavin-3,3′-digallate, thearubigins), anthocyanins (flavonals) and anthocyanidins (such as, for example, pelargonidin, peonidin, cyanidin, delphinidin, malvidin, petunidin), isoflavones (phytoestrogens) (such as, for example, daidzein (formononetin), genistein (biochanin A), glycitein), dihydroflavonols, chalcones, coumestans (phytoestrogens), and Coumestrol; Phenolic acids (such as: Ellagic acid, Gallic acid, Tannic acid, Vanillin, curcumin); hydroxycinnamic acids (such as, for example, caffeic acid, chlorogenic acid, cinnamic acid, ferulic acid, coumarin); lignans (phytoestrogens), silymarin, secoisolariciresinol, pinoresinol and lariciresinol); tyrosol esters (such as, for example, tyrosol, hydroxytyrosol, oleocanthal, oleuropein); stilbenoids (such as, for example, resveratrol, pterostilbene, piceatannol) and punicalagins.
ii) terpenes (isoprenoids) which include carotenoids (tetraterpenoids) including carotenes (such as, for example, α-carotene, β-carotene, γ-carotene, δ-carotene, lycopene, neurosporene, phytofluene, phytoene), and xanthophylls (such as, for example, canthaxanthin, cryptoxanthin, aeaxanthin, astaxanthin, lutein, rubixanthin); monoterpenes (such as, for example, limonene, perillyl alcohol); saponins; lipids including: phytosterols (such as, for example, campesterol, beta sitosterol, gamma sitosterol, stigmasterol), tocopherols (vitamin E), and γ-3, -6, and -9 fatty acids (such as, for example, gamma-linolenic acid); triterpenoid (such as, for example, oleanolic acid, ursolic acid, betulinic acid, moronic acid).
iii) betalains which include Betacyanins (such as: betanin, isobetanin, probetanin, neobetanin); and betaxanthins (non glycosidic versions) (such as, for example, indicaxanthin, and vulgaxanthin).
iv) organosulfides, which include, for example, dithiolthiones (isothiocyanates) (such as, for example, sulphoraphane); and thiosulphonates (allium compounds) (such as, for example, allyl methyl trisulfide, and diallyl sulfide), indoles, glucosinolates, which include, for example, indole-3-carbinol; sulforaphane; 3,3′-diindolylmethane; sinigrin; allicin; alliin; allyl isothiocyanate; piperine; syn-propanethial-S-oxide.
v) protein inhibitors, which include, for example, protease inhibitors.
vi) other organic acids which include oxalic acid, phytic acid (inositol hexaphosphate); tartaric acid; and anacardic acid.

In a further aspect of the first embodiment, the inventive liquid viscoelastic swallowing aid may optionally comprise at least one antioxidant. Antioxidants are molecules capable of slowing or preventing the oxidation of other molecules. The antioxidant can be any one of astaxanthin, carotenoids, coenzyme Q₁₀ (“CoQ₁₀”), flavonoids, glutathione Goji (wolfberry), hesperidin, lactowolfberry, lignan, lutein, lycopene, polyphenols, selenium, vitamin A, vitamin C, vitamin E, zeaxanthin, or any combinations thereof.

In a further aspect of the first embodiment, the inventive liquid viscoelastic swallowing aid may optionally comprise minerals. Such mineral(s) may include boron, calcium, chromium, copper, iodine, iron, magnesium, manganese, molybdenum, nickel, phosphorus, potassium, selenium, silicon, tin, vanadium, zinc, or any combinations thereof.

The optional ingredients in the inventive liquid viscoelastic swallowing aid may also include vitamin(s), such as vitamin A, Vitamin B1 (thiamine), Vitamin B2 (riboflavin), Vitamin B3 (niacin or niacinamide), Vitamin B5 (pantothenic acid), Vitamin B6 (pyridoxine, pyridoxal, or pyridoxamine, or pyridoxine hydrochloride), Vitamin B7 (biotin), Vitamin B9 (folic acid), and Vitamin B12 (various cobalamins; commonly cyanocobalamin in vitamin supplements), vitamin C, vitamin D, vitamin E, vitamin K, folic acid and biotin) essential in amounts for normal growth and activity of the body, or any combinations thereof.

Any aspects of the first embodiment as detailed above may be combined with each other.

The liquid viscoelastic swallowing aid as described herein is also intended for use in promoting safe swallowing of a Solid Oral Dosage Form (SODF) either in a healthy person or in a patient in need thereof.

In this context, “promoting safe swallowing” typically means that the bolus may be swallowed by the healthy person or a patient in need thereof preferably without making the process of swallowing “unsafe”, e.g., by aspirating the liquid viscoelastic swallowing aid and/or the bolus, choking, etc. Moreover, the liquid viscoelastic swallowing aid as described herein may also allow an improvement of the swallowing process, either in a clinical or a non-clinical manner, or both, e.g., by a reduction in discomfort, leading to an easier and more comfortable swallowing event. In other words, the term “safe swallowing” is not to be reduced to the clinical aspects of swallowing only, such as e.g., in the treatment of swallowing disorders such as dysphagia, but also concerns the administration of the inventive liquid viscoelastic swallowing aid to a healthy person as discussed further below.

A SODF as defined herein may be typically a tablet or a capsule. Usually, tablets and capsules are defined as known according to the skilled person in accordance with well-known tablet and capsule sizes as defined in the art. In this context, the capsules as preferred in the context of the current invention have a standard size of between “5” to “000” according to the definition of the FDA (see e.g., Guidance for Industry, “Size, shape and other physical attributes of generic Tablets and Capsules, U.S. Department of Health and Human Services Food and Drug Administration Center for Drug Evaluation and Research (CDER) June 2015, Pharmaceutical Quality/CMC, or https://www.fda.gov/media/87344/download, or http://www.fda.gov/Drugs/GuidanceComplianceRegulatoryInformation/Guidances/default. htm, etc.), or have a tablet length of 3 to 23 mm, preferably 3 to 22 mm, and/or a capsule length of 3 to 24 mm, preferably 3 to 22 mm.

In view of the above the invention also provides according to a second embodiment a liquid viscoelastic swallowing aid as described herein for use in promoting safe swallowing of a Solid Oral Dosage Form (SODF) in a patient in need thereof. Any of the definitions and features defined above for the liquid viscoelastic swallowing aid as described herein also apply here.

In this context, a patient in need of a treatment according to the current invention is usually a patient suffering from a swallowing disorder, or a patient suffering from dysphagia. It may also be a patient suffering from a compromised secretion of saliva, or a patient having an increased risk for choking and silent aspiration, or is a patient being prescribed multiple medications and/or suffering from multiple comorbidities, e.g. a child or an older patient suffering from multiple comorbidities and/or being prescribed multiple medications, or wherein the patient is a cancer patient, or wherein the patient suffers from Xerostomia (dry mouth), hyperglycemia, diabetes, cardiovascular disease, arthritis, hypertension, asthma, dementia, MCI, Alzheimer's disease, Parkinson's disease, epilepsy, motor neuron disease, amyotrophic lateral sclerosis (ALS), multiple sclerosis (MS), osteoporosis, stroke, chronic kidney disease, or deep vein thrombosis, or is a person having a sore throat or any further disease or condition that results in an impaired swallowing. A patient in need of such a treatment may furthermore be any patient that requires administration of a SODF as defined herein. The administration particularly allows an improvement of the swallowing process in any of the described diseases and/or allows a reduction in discomfort, leading to an easier and more comfortable swallowing event in the context of any of the herein described diseases, particularly swallowing disorders.

Such a liquid viscoelastic swallowing aid for use in promoting safe swallowing of a Solid Oral Dosage Form (SODF) in a patient in need thereof is preferably in an administrable form as described above e.g., selected from the group consisting of pharmaceutical formulations, dietary supplements, functional beverage products, food for special medical purpose (FSMP), and combinations thereof. Likewise, as said before, the liquid viscoelastic swallowing aid for use in promoting safe swallowing of a Solid Oral Dosage Form (SODF) in a patient in need thereof may be provided as described above e.g., in a concentrated form to be diluted prior to use or is provided in a ready-to-use form, or is provided as a powder to be reconstituted prior to use. Likewise, the liquid viscoelastic swallowing aid for use in promoting safe swallowing of a Solid Oral Dosage Form (SODF) in a patient in need thereof may contain an optional ingredient as described above e.g., selected from the group consisting of food additives, acidulants, buffers or agents for pH adjustment, chelating agents, colorants, emulsifiers, excipient, flavour agent, minerals, osmotic agents, a pharmaceutically acceptable carrier, preservatives, stabilisers, sugar(s), sweetener(s), texturiser(s), vitamin(s), proteins, lipids and and/or carbohydrates. Any of the features defined before for the first embodiment also apply for this second embodiment.

Moreover, according to a third embodiment the invention also provides a preferably non-therapeutic use of a liquid viscoelastic swallowing aid as described herein for promoting safe swallowing of a Solid Oral Dosage Form (SODF) in a healthy person. Any of the definitions and features defined above for the liquid viscoelastic swallowing aid as described herein also apply here. Such a non-therapeutic use preferably leads to an improvement of the swallowing process by a reduction in discomfort, leading to an easier and more comfortable swallowing event.

Generally, such a healthy person is preferably a person that does not suffer from a disease, preferably that does not suffer from any of such diseases as described before, more preferably that does not suffer from any swallowing disease, as described before. Such a healthy person may prefer using a liquid viscoelastic swallowing aid as described herein e.g., to avoid an uncomfortable experience when swallowing such an SODF. SODF in this context may e.g., be common nutritional supplements, vitamins, etc., but optionally may also be common medicaments, such as tablets against pain, headache, migraine, menstrual pain, back pain, etc. or may be medications prescribed for short or long-term treatments, such as hypertension, hyperthyroidism, hashimoto, immunomodulating medicaments, antidepressiva, psychopharmaca, antiepileptika, antibodytherapies, antibiotics, etc. Accordingly, a healthy person in this context may also be an otherwise healthy person, that receives single or multiple medications, e.g. from the above medications, but does not suffer from any of the above described swallowing diseases. It also may be an otherwise healthy person that has e.g., a sore throat, but preferably does not suffer from a swallowing problem, etc. Healthy persons may also be persons which ingest simple nutritional supplements, such as e.g. vitamins, minerals, fatty acids such as polyunsaturated fatty acids and omega-3-fatty acids, folic acid,

The terms “patient in need thereof” as well as “healthy person” both refer to any human, animal, mammal that can benefit from the liquid viscoelastic swallowing aid as described herein. It is to be appreciated that animal includes, but is not limited to, mammals. Mammal includes, but is not limited to, rodents, aquatic mammals, domestic animals such as dogs and cats, farm animals such as sheep, pigs, cows and horses, and humans.

Such a liquid viscoelastic swallowing aid for a non-therapeutic use in a healthy person may be preferably in an administrable form as described above, e.g., selected from the group consisting of pharmaceutical formulations, dietary supplements, functional beverage products, food for special medical purpose (FSMP), and combinations thereof. Likewise, as said before, the liquid viscoelastic swallowing aid for a non-therapeutic use in a healthy person may be provided e.g., in a concentrated form to be diluted prior to use or is provided in a ready-to-use form, or is provided as a powder to be reconstituted prior to use. Likewise, the liquid viscoelastic swallowing aid for a non-therapeutic use in a healthy person may contain an optional ingredient as described above e.g., selected from the group consisting of food additives, acidulants, buffers or agents for pH adjustment, chelating agents, colorants, emulsifiers, excipient, flavour agent, minerals, osmotic agents, a pharmaceutically acceptable carrier, preservatives, stabilisers, sugar(s), sweetener(s), texturiser(s), vitamin(s), proteins, lipids and and/or carbohydrates. Any of the features defined before for the first embodiment also apply for this third embodiment. Moreover, aspects of the third and the fourth embodiment may be combined.

Furthermore, according to a fourth embodiment the invention is also directed to a method of administering or feeding the liquid viscoelastic swallowing aid as described herein to an individual for promoting swallowing of a Solid Oral Dosage Form (SODF). The liquid viscoelastic swallowing aid as described herein for the first embodiment. An individual may be either a healthy person and/or a patient to be treated as defined above.

Accordingly, the invention is also directed to a method of administering or feeding the liquid viscoelastic swallowing aid as described herein to an individual for promoting swallowing of a Solid Oral Dosage Form (SODF), comprising:
a. providing the liquid viscoelastic swallowing aid as described herein;
b. providing a Solid Oral Dosage Form (SODF), preferably as described herein;
c. mixing the Solid Oral Dosage Form (SODF) as defined herein and the liquid viscoelastic swallowing aid as defined herein, e.g., on a spoon, forming a bolus to be swallowed; and
d. administering the bolus to a patient in need thereof.

Alternatively, the invention is also directed to a method of administering or feeding the liquid viscoelastic swallowing aid as described herein to an individual in need thereof for promoting swallowing of a Solid Oral Dosage Form (SODF), comprising:
a. providing the liquid viscoelastic swallowing aid as described herein;
b. providing a Solid Oral Dosage Form (SODF), preferably as described herein;
c. administering the Solid Oral Dosage Form (SODF) as described above to a patient in need thereof without swallowing the SODF;
d. administering the liquid viscoelastic swallowing aid as described above, thereby mixing the Solid Oral Dosage Form (SODF) as defined herein and the liquid viscoelastic swallowing aid as defined herein, preferably in the mouth, forming in situ a bolus to be swallowed.

Alternatively, the invention is also directed to a method of administering or feeding the liquid viscoelastic swallowing aid as described herein to an individual in need thereof for promoting swallowing of a Solid Oral Dosage Form (SODF), comprising:
a. providing the liquid viscoelastic swallowing aid as described herein;
b. providing a Solid Oral Dosage Form (SODF), preferably as described herein;
c. administering the liquid viscoelastic swallowing aid as described herein to a patient in need thereof without swallowing the liquid viscoelastic swallowing aid;
d. administering the Solid Oral Dosage Form (SODF) as described above, thereby mixing the Solid Oral Dosage Form (SODF) as defined herein and the liquid viscoelastic swallowing aid as defined herein, preferably in the mouth, forming in situ a bolus to be swallowed.

It should be appreciated that the various aspects and embodiments of the detailed description as disclosed herein are illustrative of the specific ways to make and use the invention and do not limit the scope of invention when taken into consideration with the claims and the detailed description. It will also be appreciated that features from aspects and embodiments of the invention may be combined with further features from the same or different aspects and embodiments of the invention.

As used in this detailed description and the appended claims, the singular forms “a,” “an” and “the” include plural referents unless the context clearly dictates otherwise.

All ranges described are intended to include all numbers, whole or fractions, contained within the said range.

### FIGURES

FIG. 1: shows a scheme of the in vitro setup used to replicate the oral phase of swallowing, adapted from Marconati and Ramaioli (2020)

FIG. 2: shows steady shear viscosity measurements of the different liquids investigated in the Experiments as carriers

FIG. 3: shows a filament thinning of (a) Beta-glucan sample L1, (b) ThickenUp Clear (TUC) L1, (c) polyethylene glycol (PEO) L1, (d) Glycerol L1, (e) Beta-glucan sample L3, (f) TUC L3, (g) PEO L3, (h) Glycerol L3, (i) Gloup L4, (j) TUC L4. Representative pictures of each liquid carrier at t 0, t ¼ breakup, t ½ breakup, t ¾ breakup, and t breakup (value of t breakup for this specific sample is indicated on the image).

FIG. 4: shows the evolution of the filament midpoint diameter in time, up to t breakup, for (a) TUC, glycerol, and Gloup samples, and (b) the beta-glucan compositions, and PEO samples. Mean values are presented, and error bars are not displayed to improve clarity.

FIG. 5: shows extensional relaxation times of the liquid carriers tested herein in relation to their shear viscosity at γ=50 s⁻¹.

FIG. 6: shows snapshots of representative in vitro swallows (capsules and tablets).

FIG. 7: shows characteristic oral transit time t TO measured in vitro for the different liquid carriers alone, with a capsule, or with a tablet. Water TO is taken as reference (vertical line marked with an asterix).

FIG. 8: shows calculated volumes of residues left in the plastic membrane simulating the oral cavity after in vitro swallowing. Water residues are taken as reference (vertical line marked with an asterix).

FIG. 9: shows bolus elongation at t TO (bolus length expressed as a percentage of the initial size of the bolus at to). Water is taken as reference (vertical line marked with an asterix)

FIG. 10: shows a quantification by image analysis of the relative position of the capsule/tablet with respect to bolus front at TO

FIG. 11: shows the position of the SODF during in vitro swallowing with different liquid carriers: capsules in water and L1 fluids (a), in L3 and L4 fluids (b), and tablets in water and L1 fluids (c), in L3 and L4 fluids (d)

### EXAMPLES

### 1. Materials and Methods

### 1.1 Materials

The inventive examples considered mineral water (Vittel) and five different types of liquid carriers (three thickener solutions and two model systems). Different concentrations were used for each carrier with the objective to obtain two categories of fluids, classified as Level 1 and Level 3 to 4 according to the International Dysphagia Diet Standardization Initiative (IDDSI) framework. Traces of a dye (0.02% w/w) were added to the samples to enhance image contrast.

Beta-glucan samples were provided by Nestle Research (Lausanne, CH). The frozen beta-glucan samples were thawed in a refrigerator at 4° C. for 18 hours, then left to equilibrate at ambient temperature for 3 hours, prior to the rheological characterization and in vitro tests. Beta-glucan samples serve as a model for extensional viscous carriers as claimed, namely beta-glucans, plant-derived mucilage and/or a plant-extracted gum.

Aqueous suspensions of a commercial xanthan gum-based thickener (Resource® ThickenUp™ Clear, Nestle Health Science, commercially available), referred to as TUC in the following text, were also used. Suspensions with different IDDSI levels were prepared by adding 100 mL of mineral water to 0.6 g, 2.4 g, or 3.6 g of TUC powder, according to the recommendations of the supplier. TUC is commonly used in the management of dysphagia and was used as an example of commercial texture modifier, readily available in local pharmacies.

The swallowing aid “Gloup original”, with a strawberry/banana flavor, was also tested (Rushwood B.V., Raamsdonksveer, NL). This product is proposed as a swallowing gel for medicines, and contains carrageenans. The gel was directly poured from the 150 mL container at room temperature.

Two model fluids, with limited rheological complexity compared to the two previous food systems, were also considered in this study. First, aqueous suspensions (1 and 3% w/w in mineral water) of polyethylene oxide (PEO, CAS 25322-68-3, average molecular weight M_{w}=10\hat{ }6 g/mol) were used to further investigate the effect of elasticity. The polymer was left hydrating overnight in sealed containers under magnetic stirring. Finally, solutions of glycerol (Sigma-Aldrich, CAS Number 56-81-5) were used. Glycerol was diluted with mineral water to obtain an IDDSI level 1 mixture (72.8% glycerol w/w), and an IDDSI level 3 mixture (98.8% glycerol w/w).

Several shapes and sizes of SODF may be available for the same medication and dosage. Tablets and capsules sizes may range from 3 to 25 mm in length according to Jacobsen et al. (Jacobsen et al. 2016), but people tend to be more comfortable with round, white, medium-sized (between 8 and 12 mm in diameter) coated tablets (Fields et al. 2015; Overgaard et al. 2001; Radhakrishnan 2016). Therefore, a large uncoated dark tablet and a HPMC capsule equivalent in size were selected (Table 1). Spirulina supplements available in these two formats were sourced from Anastore, “Spiruline Biologique”, 500 mg, (see e.g. https: www.anastore.com/fr/articles/NA40 spirline bio.php) and Vegavero (“Spirulina Bio”, 1,000 mg, https://shop.vegavero.com/uk/p/Spirulina-Organic).

TABLE 1
Characteristics of the SODF used in the current experiments.

| | | | Aspect | Tablet to bolus | Calculated | | |
|---|---|---|---|---|---|---|---|
| SODF | Shape | Size | ratio | cross section | volume | Mass | Density |
| Capsule | Size 0 | 22 mm long | 2.93 | 24.1% | 861 mm³ | 0.59 g | 0.7 |
| “Spiruline | | 7.5 mm width | | | | | |
| Biologique” | | | | | | | |
| Tablet | oblate, | 22 mm long | 3.14 | 26.7% | 842 mm³ | 1.02 g | 1.2 |
| “Spirulina | scored | 7 mm width | | | | | |
| Bio” | | | | | | | |

### 1.2. Methods

### 1.2.1 IDDSI Flow Test

The IDDSI flow test was run at room temperature in triplicate to evaluate the IDDSI level of each liquid carrier (IDDSI 2019). In this test, a standard luer slip tip syringe is filled up to the 10 mL mark with the sample, and the liquid is then allowed to flow for 10 s. Based on the remaining volume left in the syringe, liquid samples are categorized in four levels of increasing thickness: Level 0 (less than 1 mL remaining), Level 1 (1-4 mL remaining), Level 2 (4-8 mL remaining), Level 3 (no less ten 8 mL remaining). If the liquid does not flow through the tip of the syringe, it is classified as Level 4. IDDSI Level 4 liquids can also be evaluated with the IDDSI spoon tilt test: they must hold their shape on a spoon and fall off easily if the spoon is tilted.

### 1.2.2. Steady Shear Tests

The shear viscosity was assessed with a Modular Compact Rheometer (MCR) 102 (Anton Paar GmbH, Graz, Austria), at 25° C. A cone and plate geometry (diameter=50 mm, cone angle=4°, truncation=500 μm), and a 0.5 mm gap were used to obtain flow curves in a range of shear rates between 0.5 and 800 reciprocal seconds. Three repetitions were performed for each sample.

### 1.2.3. Extensional Properties

The extensional properties of the samples were measured by capillary break-up rheometry using a HAAKE CaBER 1 (Thermo Electron, Karlruhe, Germany) at room temperature (preferably 25° C.). The initial separation between the two circular plates (6 mm in diameter) was set at 3 mm, and an axial displacement up to 10 mm was imposed in 50 ms to drive the filament thinning. The evolution in time of the midpoint diameter of the thread was measured with a laser micrometer with a beam thickness of 1 mm and a resolution of 20 μm. The extensional relaxation time was calculated with the CaBER Analysis software (Haake RheoWin Software, version 5.0.12) by fitting the data with the elastic (exponential) model. Five repetitions were performed for each sample. High-speed videos of the experiments were also taken at 1,000 frames per second to record the shape evolution of the capillary thread using a Phantom V1612 high-speed camera (Vision Research, Wayne, NJ).

### 1.2.4. In Vitro Swallowing

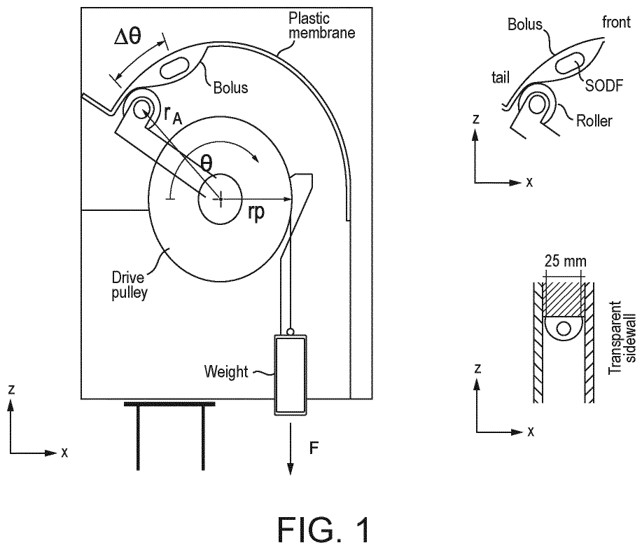

The effect of the rheological properties of the different liquid carriers on the dynamics of SODF swallowing was investigated in vitro with an experimental setup (FIG. 1) that considers the peristaltic motion induced by the tongue during the oral phase of swallowing. A comprehensive description of this experimental setup, the discussion of the limitations and the validation against ultrasonic in vivo measurements has already been presented by Mowlavi et al., (2016).

The capsule or tablet was first positioned in the dry plastic membrane, and aligned with its longitudinal axis. Thus, the smallest cross-section of the SODF was in the direction of the flow. Then, 4.5 mL of liquid carrier was carefully pushed in and after 2 min the roller movement was triggered. This contact time between SODF and liquid was controlled in order to limit the dissolution of the capsule/tablet before swallowing (see FIG. 1).

The instantaneous position of the bolus and the SODF during the in vitro swallowing experiment was recorded using a high-speed camera (model ac1920-155 mm, Basler, Ahrensburg, Germany) at 200 frames per second. The mass of residues left inside the plastic membrane after a swallow was also recorded for each experiment. At least three repetitions were performed for each set of experimental variables.

The time at which the front of the bolus (FO) exits the plastic membrane, and the time at which the tail of the bolus (TO) leaves the membrane were identified on the video recordings of each experiment. In this experimental setup, the plastic membrane plays the role of the oral cavity, therefore FO and TO are considered as characteristic oral transit times.

Image processing tools (ImageJ and GNU Octave) were used to extract the instantaneous position of the roller (corresponding to the bolus tail), and the SODF center of mass during the swallowing experiment up to TO.

The bolus length was measured between the roller and the bolus front at to, FO and TO, and expressed as a percentage of the initial size of the bolus at to. Similarly, the position of the SODF in the bolus was quantified by measuring the distance between the SODF front and the bolus front at to, FO and FO (A front).

Additionally, the difference in the angular position of the center of mass of the SODF and the angular position of the roller was followed up to FO:

Δθ=θ_{SODF}−θᵣₒₗₗₑᵣ (1)

A decreasing Δθ indicates that the SODF was slower than the liquid carrier and moved towards the tail of the bolus, and inversely an increasing Δθ shows that the SODF was flowing faster that the liquid and was moving toward the front of the bolus.

### 1.2.3. Statistical Analysis

The results are shown in terms of the mean f the standard deviation. The statistical significance of the results was tested using one-way analysis of variance (ANOVA) and differences between group means were analyzed by Tukey's multiple comparison test with a probability level of 0.05 (p<0.05). Statistical analysis was carried out with Origin 2020b (OriginLab Corporation, Northampton, MA).

### 1.3. Results and Discussion

### 1.3.1. IDDSI Flow Test

The set of liquid carriers considered in this study was designed to obtain two different categories of consistencies: water and thin liquids on one side, and thicker liquids adapted for individuals with dysphagia on the other side.

The consistency of each liquid carrier was first qualitatively evaluated according to the IDDSI framework (Table 2).

TABLE 2
Classification of the liquid carriers used in this
study according to the IDDSI testing methods (flow
test and spoon tilt test), at room temperature.

| | Flow test | | |
|---|---|---|---|
| | Volume | Inter- | |
| | remaining | pretation | |
| | (mL) in | (IDDSI | |
| | the syringe | classi- | Abbreviated |
| Carrier | after 10 s | fication) | name |
| Mineral water | 0 | 0 | Water |
| Beta-glucan sample 0.3% | 2.75 | 1 | Beta-glucan |
| (w/w) | | | sample L1 |
| Beta-glucan sample 1% | 9.00 | 3 | Beta-glucan |
| (w/w) | | | sample L3 |
| ThickenUp Clear 0.6% (w/v) | 1.25 | 1 | TUC L1 |
| ThickenUp Clear 2.4% (w/v) | 9.50 | 3 | TUC L3 |
| ThickenUp Clear 3.6% (w/v) | 10 | 4 | TUC L4 |
| Gloup Original | 10 | 4 | Gloup L4 |
| PEO 1% (w/w) | 1.75 | 1 | PEO L1 |
| PEO 3% (w/w) | 9.50 | 3 | PEO L3 |
| Glycerol 72.8% (w/w) | 1.50 | 1 | Glycerol L1 |
| Glycerol 98.8% (w/w) | 9.50 | 3 | Glycerol L3 |

Apart from water, three groups of samples were obtained. The beta-glucan sample 0.3% (w/w), and the suspensions of TUC 0.6% (w/v), PEO 1% (w/w), and glycerol 72.8% (w/w) were classified as IDDSI Level 1. The beta-glucan sample 1% (w/w), and the suspensions of TUC 2.4% (w/v), PEO 3% (w/w), and glycerol 98.8% (w/w) were classified as IDDSI Level 3. Gloup Original and TUC 3.6 (w/v) were classified as IDDSI Level 4.

Gloup Original is marketed as an IDDSI Level 3 product, but it was classified here as IDDSI Level 4 since no outflow was measured in the 10 s test-time. This classification was confirmed with the IDDSI spoon tilt test. (Malouh et al. 2020) also classified this product as Level 4 when directly poured from the bottle.

### 1.3.2. Rheological Properties

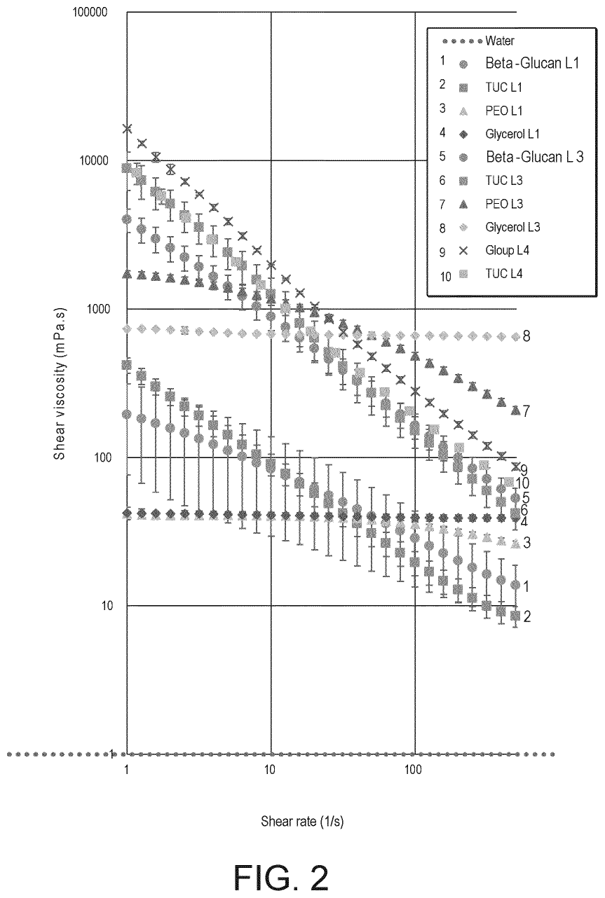

Flow curves obtained in steady shear are presented in FIG. 2. Overall, the samples showed a shear thinning behavior, except for the mineral water and the glycerol solutions which are Newtonian fluids (FIG. 2). However, specific differences were observed.

TUC suspensions had a pronounced shear thinning behavior across this range of shear rates, independently of the concentration used, while PEO suspensions were less shear thinning, suggesting a viscosity plateau at low shear rates. The extent of this viscosity plateau decreased when increasing the polymer concentration (up to 100 s−1 for PEO L1, and up to 1 s⁻¹ for PEO L3). Compared to TUC and PEO, the beta-glucan samples had an intermediate shear thinning behavior. Similar results were reported by Marconati and Ramaioli (2020).

The flow curve of Gloup showed a strong shear thinning behavior too, as it can be expected for a product composed of carrageenan. Across the range of shear rates considered, Gloup, TUC L3, and TUC L4 had similar viscosities.

The four IDDSI Level 1 carriers had comparable shear viscosities at γ=50 s−1 (Appendix). TUC L1 had the lowest (30.76±3.12 mPa·s), and the beta-glucan sample L1 had the highest (40.09±13.01 mPa·s). To provide the reader with a benchmark, commercial orange juices have similar viscosities (Marconati et al. 2018). In contrast, shear viscosities at γ=50 s⁻¹ differed significantly between IDDSI Level 3 liquid carriers. Two groups were observed: beta-glucan sample L3 and TUC L3 were lower in viscosity than PEO L3 and glycerol L3 (approx. 275 and 670 mPa·s, respectively).

The shear rheology of texture modifiers is commonly reported at shear rates of 50 reciprocal seconds, which facilitates comparison between studies. However, it has been established that shear rates for the whole swallowing process can vary from 1 s−1 in the mouth and the esophagus to 1,000 s⁻¹ in the pharynx (Gallegos et al. 2012; Nishinari et al. 2016).

According to FIG. 2, liquid carriers with the same IDDSI level had different viscosities at low and high shear rates (i.e., ≤10 s⁻¹ and ≥100 s⁻¹, respectively), except for TUC suspensions and Gloup which are both similar, strongly shear thinning products. These results suggest that IDDSI levels represent different viscosity ranges if the fluids considered are Newtonian, slightly shear thinning or strongly shear thinning.

### 1.3.3. Extensional Properties

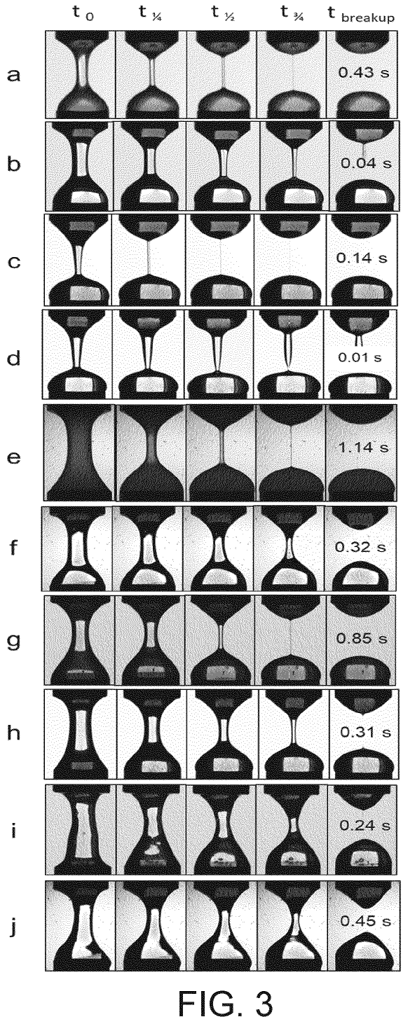

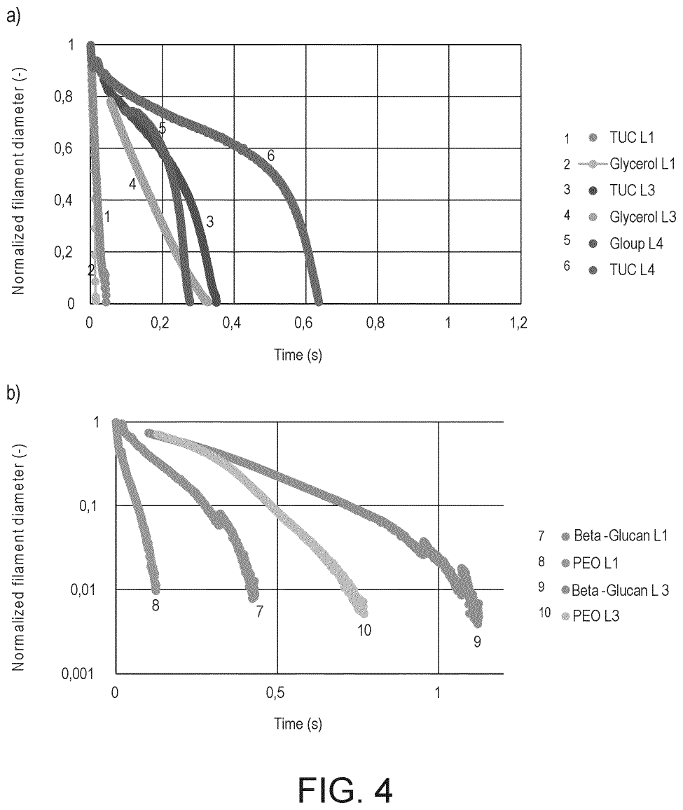

The extensional properties of the liquid carriers were studied by Capillary Breakage Extensional Rheometry (CaBER). Selected images extracted from video recordings of the transient filament thinning until break-up for each sample are presented in FIG. 3, and the temporal evolution of the midpoint filament diameter, normalized by the initial midpoint diameter is illustrated in FIG. 4.

Different regimes of capillary thinning and break-up were observed, independently of the IDDSI level of the carrier. For TUC suspensions, Gloup, and glycerol solutions, the filament had a hour-glass shape (FIG. 3 b, d, f, h, i, j). The filament was rapidly evolving in time and short break-up time were measured (i.e., ≥0.5 s). For glycerol samples, the filament diameter decreased linearly in time, which is typically observed for Newtonian fluids (Anna and McKinley 2000). For TUC and Gloup, an acceleration of filament break-up in a viscous dominated regime was observed, characteristic of shear thinning liquids (McKinley n.d.) (FIG. 4a).

In contrast, the liquid bridge formed by PEO suspensions and the beta-glucan samples was cylindrical (FIG. 3 a, c, e, g). In this case, the radius of the cylindrical capillary decreased exponentially in time and larger break-up time were registered (FIG. 4b). This behavior is distinctive of elastic fluids (Anna and McKinley 2000).

Such elastic dominated regimes can be described by a extensional relaxation time (k) (Arnolds et al. 2010). In the experimental conditions of this study, the beta-glucan samples had larger k than the PEO suspensions (0.04 to 0.10, and 0.01 to 0.07, respectively. Similar results were obtained by Marconati and Ramaioli (2020).

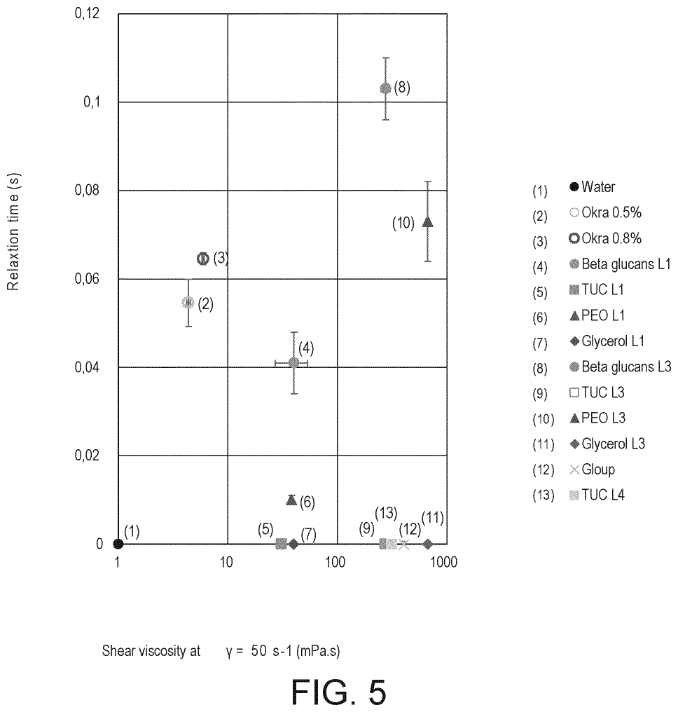

Overall, larger break-up times were measured for IDDSI level 3 carriers compared to IDDSI level 1 samples. At higher thickener concentrations, the contribution of the viscous drainage on the filament thinning dynamics increased. This was also observed for the elastic liquid carriers, but in this case, k also increased when increasing the polymers concentrations (FIG. 5). Interestingly, for the beta-glucan samples k values increased rapidly with concentration while the increase in shear viscosity was moderate (FIG. 5). These samples may therefore be considered as elastic thin fluids.

Values as displayed in FIG. 5 regarding rheological properties of liquid carriers characterized using shear and extensional rheometry are as follows:

| | Shear viscosity | Single relaxation |
|---|---|---|
| Carrier | γ = 50 s−1 (mPa · s) | time (s) |
| Water | 1.00 ± 0.00 | — |
| Okra 0.5% | 4.37 ± 0.16 | 0.055 ± 0.005 |
| Okra 0.8% | 5.94 ± 0.13 | 0.065 ± 0.001 |
| Beta glucans L1 | 40.09 ± 13.01 | 0.041 ± 0.006 |
| TUC L1 | 31.72 ± 2.76 | — |
| PEO L1 | 36.72 ± 2.40 | 0.010 ± 0.001 |
| Glycerol L1 | 39.77 ± 1.95 | — |
| Beta glucans L3 | 275.39 ± 26.66 | 0.103 ± 0.007 |
| TUC L3 | 272.97 ± 29.30 | — |
| PEO L3 | 674.71 ± 20.17 | 0.068 ± 0.003 |
| Glycerol L3 | 669.32 ± 5.22 | — |
| Gloup | 401.20 ± 8.46 | — |
| TUC L4 | 319.85 ± 7.55 | — |

Values also include rheological properties for okra (0.5% and 0.8%), which were measured as described above for beta-glucan samples. As can be seen from FIG. 5, all the claimed data points for compounds contained in the inventive liquid viscoelastic swallowing aid can be matched, particularly shear viscosity values and single relaxation times. IDDSI levels can also be arrived at within the claimed values.

### 1.3.4. SODF In Vitro Swallowing

The in vitro experiments aimed at understanding the effect of liquid carriers with different rheological properties on the swallowing dynamics of capsules and tablets.

Bolus velocity, post-swallow residues, bolus elongation, and position of the SODF in the bolus were first investigated with water, considered as a reference.

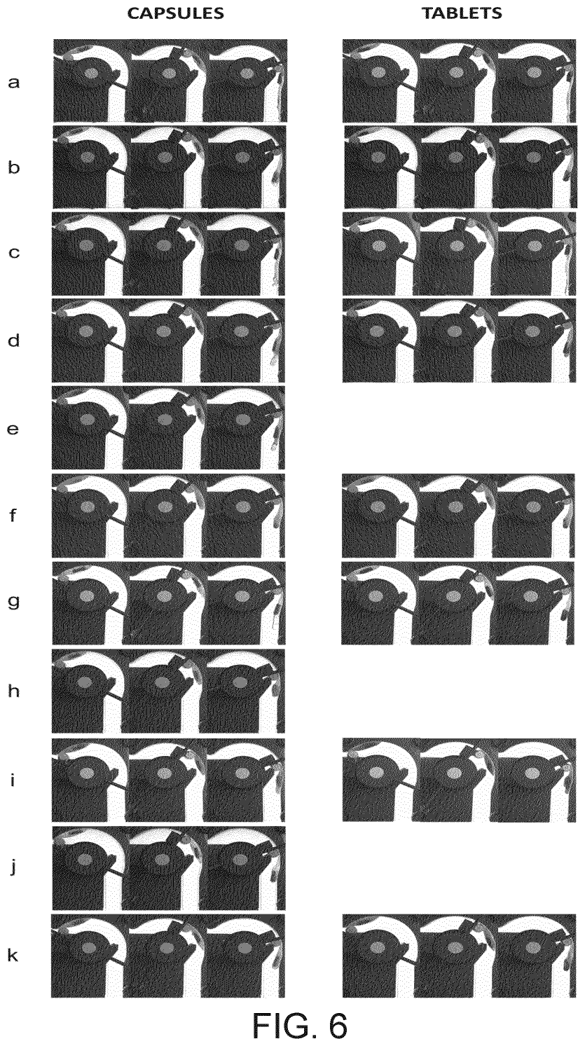

Snapshots from the experimental video recordings are presented in FIG. 6. These pictures were taken at the beginning of the experiment (to), when the front of the bolus reached the end of the simulated oral cavity (FO), and when the tail of the bolus exited the simulated oral cavity (TO).

### 1.3.5. Oral Transit Times

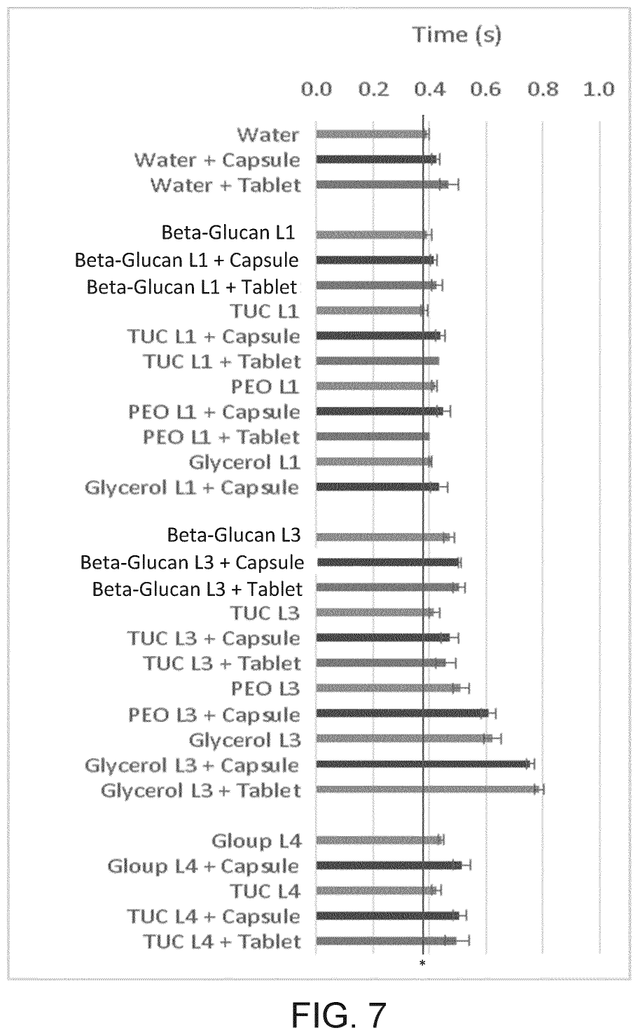

Characteristic oral transit times for the different sets of carriers and SODF are presented in FIG. 7.

With water, t FO was not modified by the presence of SODF in the bolus, but t TO was slightly delayed, meaning that capsules and tablets both slowed down bolus ejection (delay of 0.03 and 0.06 s, respectively) (FIG. 7). These results suggest that large SODF only slightly influence bolus velocity when swallowed with water.

All tests performed with L1 liquids with and without SODF led to similar TO to water (without SODFF). When compared to water L1 liquids were therefore all able to avoid the slowing down induced by the presence of a capsule.

The beta-glucan sample L3, Gloup, and TUC L4, only slightly delayed t FO and t TO compared to water (FIG. 7), while TUC L3 showed a transit time similar to water Glycerol L3 showed significantly higher FO and TO. The oral transit time of the tablet with glycerol L3 was the longest of all the samples tested and reached 0.79 s, which is almost twice the transit time with water (FIG. 7). This delay is attributed to the relatively high viscosity of this Newtonian sample at high shear rates (approx. 650 mPa·s at γ≥50 s⁻¹).

When swallowed with any IDDSI level 3 or 4 liquid carrier, both SODF delayed t TO by 0.05 to 0.2 s, following this increasing order in delay: TUC and Gloup<beta-glucan sample<PEO<glycerol (FIG. 7). This seems to be related to the shear viscosity of the carriers at γ=300 s⁻¹. No differences were observed between capsules and tablets.

This suggest that the impact of the SODF on the oral transit time also depends on the rheological properties of the liquid carriers at high shear rates. In other words, delays increase with increasing high shear rates viscosities. This can be explained by the small gaps present around the ODF during the flow, where high shear rates can be reached.

These results are consistent with a previous study (Marconati et al. 2018) in which longer transit times, higher variability and lower bolus velocities were registered for large SODF (prolate spheroids, equivalent in volume to a d=10 mm sphere) in glycerol and orange juice (viscosity=1.05±0.05 Pa·s and 0.03±0.01 Pa·s, respectively).

### 1.3.6. Post-Swallow Residues

The mass of residues left in the plastic membrane was measured after each swallow.

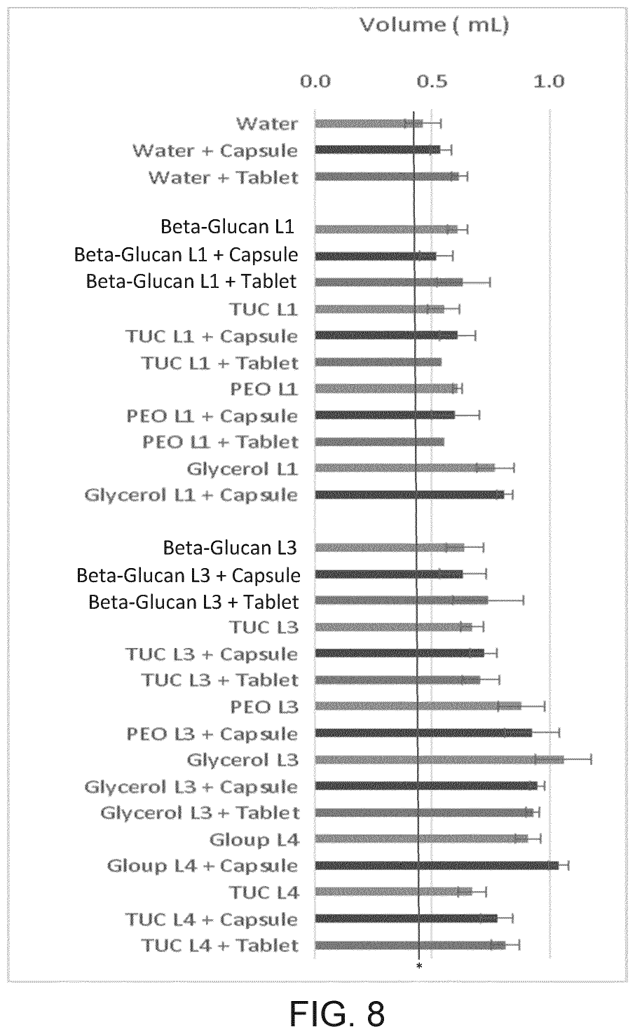

With water, post-swallow residues were increased by the presence of the tablet in the bolus (FIG. 8). This was probably related to the fast dissolution of the uncoated tablet in water since traces of dark residues were observed in the membranes.

Overall, the amount of post-swallow residues increased with the shear viscosity of the samples and no clear effect of the SODF on post-swallow residues was observed (FIG. 8). Among the IDDSI level 1 carriers, the glycerol solution left more residues (approx. 0.8 mL) than the other liquid carriers (between 0.5 and 0.6 mL). For beta-glucan samples and TUC, no significant effect of the concentration was observed. In contrast, the post-swallow residues were significantly higher for PEO and glycerol L3 compared to the lower concentration solutions classified as IDDSI L1, and reached approx. 0.9 and 1 mL which is twice the volume of residues measured with water. Gloup left also an important amount of post-swallow residues in the membrane (0.9 to 1 mL, equivalent to glycerol L3).

Excessive oropharyngeal residues can cause discomfort (i.e., unpleasant feeling that the bolus sticks in the throat), and multiple swallows can be necessary to clear the residues, which may decrease the palatability of a product. Residues can also lead to aspiration by people suffering from swallowing disorders and result in respiratory complications, such as pneumonia. Therefore, when developing swallowing aids, care must be taken to avoid the adverse effects of increased viscosity on residues and palatability. Xanthan gum-based thickeners, like TUC, are often preferred to starch-based thickeners in the management of dysphagia because they improve the swallowing safety without increasing the oropharyngeal residues (Hadde et al. 2019; Ortega et al. 2020; Rofes et al. 2014). Just as TUC, the beta-glucan samples evaluated in this study resulted in limited in vitro post-swallow residues. Clinical results may confirm a positive impact for people with dysphagia.

### 1.3.7. Bolus Elongation

The length of the bolus was evaluated by image analysis at to, t FO, and t TO, for each set of liquid carrier and SODF. At to, bolus length was 43.1±0.8 mm without SODF, 47.0±1.2 mm with capsules, and 47.2±1.4 mm with tablets. The presence of capsules and tablets in the bolus increased its volume, resulting in a longer initial bolus and in a higher risk of pre-swallow leakages, especially with water and IDDSI level 1 fluids.

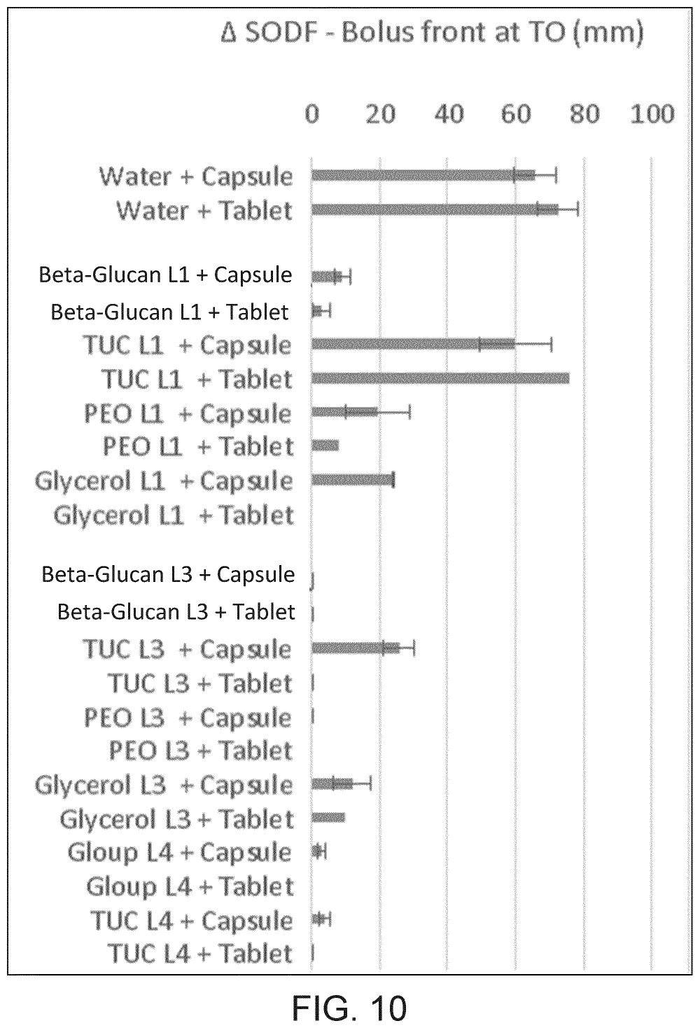

At t FO, for SODF swallowed with water or TUC L1, an increase in bolus length was observed (FIG. 9). This is attributed to liquid leakages before the experiment was triggered. In contrast, a decrease in bolus length was noticed for the most viscous samples (e.g. PEO and glycerol L3), which may be related to the partial loss of carrier during swallowing (i.e., left as residue in the membrane) (FIG. 9).

In this in vitro experiment, the liquid bolus ejected from the plastic membrane is subject to gravitational acceleration, which induces bolus elongation, and to die swell in the case of viscoelastic liquid (i.e., bolus expansion). The shear viscosity of the liquid carrier and the interaction between both phenomena will determine the bolus shape at t TO.

Water swallows resulted in long boluses at t TO (FIG. 6a and FIG. 9). Bolus length was almost doubled between to and t TO, and the presence of SODF increased bolus elongation even further. This is not desirable for patients with dysphagia because stretched boluses are more likely to break during swallowing and may increase the risk of aspiration in vivo (Hadde et al. 2019).

Results similar to water were observed with TUC L1 (bolus elongation >175%, increased by the presence of SODF). Shorter boluses were measured for the other IDDSI level 1 liquid carriers (beta-glucan sample, PEO, and glycerol), with no significant differences in bolus length when swallowing the SODF (FIG. 9).

All IDDSI level 3 fluids had shorter boluses at t TO, compared to water (FIGS. 6 and 9), and no significant effect of the SODF was observed. PEO L3 samples resulted in the lowest bolus elongation values (80 to 85%) and TUC L3 samples in the highest bolus elongation values (120 to 140%). Bolus elongation was also limited for Gloup and TUC L4, and was about 105% for both carriers (FIGS. 6 and 9).

These results suggest that bolus elongation at the exit of the oral cavity is related to the viscosity of the liquid carriers at high shear rates (BL of L3 fluids <BL of L1 fluids), and to the extensional properties of the liquid carriers (BL of beta-glucan sample L1 similar to BL of TUC L3).

A compact bolus shape was suggested as a way to promote a smoother and more controlled bolus flow through the pharynx based on videofluoroscopy observations (Hadde et al. 2019) This parameter should be further investigated in vivo, to evaluate the impact of a broader range of extensional and viscoelastic properties. 1.3.8. Position of the SODF As can be seen in FIG. 6, the position in the bolus of capsules and tablets varied according to the liquid carrier used. In order to examine this phenomenon in more detail, the relative position of the SODF with respect to bolus front was quantified from the videos of the experiments at to, FO, and TO (FIG. 1).

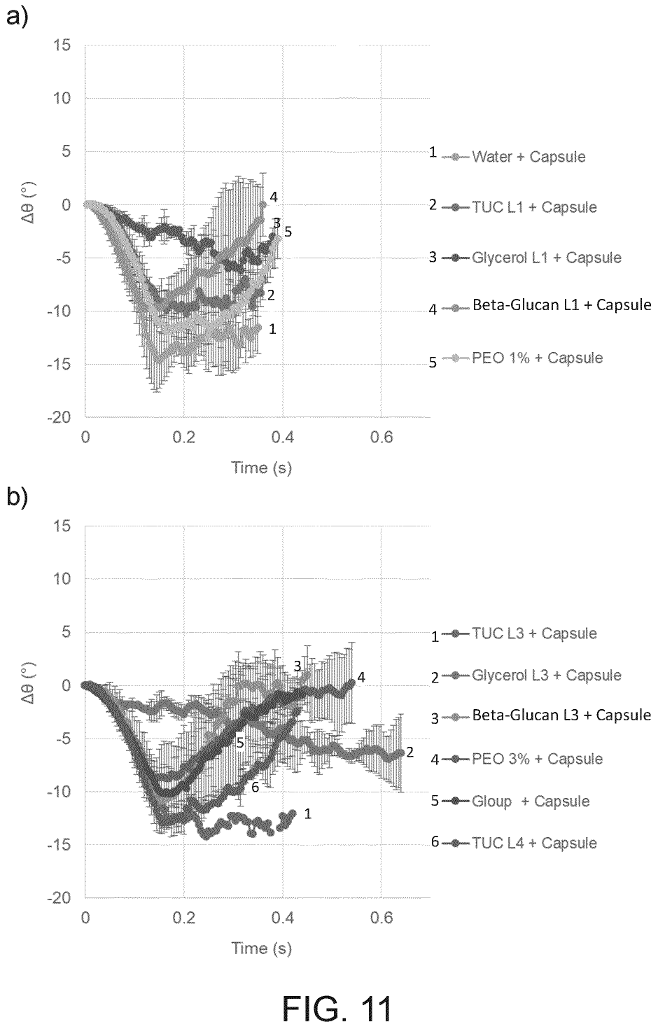

Before the swallow, the position of the SODF depended on its buoyancy in the liquid carrier. In water, the low density (0.7 g/mL) of the capsules led to floating, and to positioning close to the front of the bolus (FIGS. 6 and 10). In contrast, the tablet (density of 1.2 g/mL) settled out and positioned close to the tail of the bolus (FIGS. 6 and 10). Similar results were observed with the IDDSI Level 1 carriers. But with IDDSI L3 fluids, tablets were found in the middle of the bolus, except with the beta-glucan sample.

All the liquid carriers used in this study had a density of approx. 1.0 g/mL, except the glycerol solutions, which had a density of 1.2 g/mL. So, glycerol solutions and tablets had the same density; the tablets did not sediment and had the same position than capsules at to in glycerol boluses.

When swallowed with water, both SODF lagged toward the bolus tail during in vitro swallowing (FIGS. 6 and 10). Under the imposed squeezing action of the roller, water was able to flow through the gap present around the SODF, leading to the solid lagging behind (Marconati et al. 2018). Capsules and tablets entered the simulated pharynx after the bulk of the liquid, with no liquid left to help transport them out.

This phenomenon has already been reported by Marconati et al., (2018) in a similar in vitro experiment with model large spherical tablets in orange juice.

These results suggest that water is not an efficient carrier for capsules and tablets. It flows faster than the SODF, which lags behind. Multiple swallows or larger volumes of water may then probably be needed to transport the SODF from the oral cavity to the esophagus, which multiply the risks for patients with dysphagia (Hey et al. 1982; Stegemann et al. 2012; Yamamoto et al. 2014). Actually, in vivo studies have shown that when placebos could not be swallowed at the first attempt, they remained mainly in the mouth of the patients (Schiele et al. 2015; Yamamoto et al. 2014).

Comparable results were obtained with TUC L1. The liquid bolus was stretched and the SODF was close the bolus tail at t FO and at t TO (FIGS. 6 and 10). But differences were observed with the other IDDSI level 1 liquid carriers. At t TO, in PEO and glycerol (L1), capsules were positioned in the middle of the liquid bolus, and in the beta-glucan sample both capsules and tablets were found at the front of the liquid bolus (FIGS. 6 and 10). This is considered as an improvement in the transport of the SODF because it suggests that the solid may be efficiently embedded in the carrier during the whole swallowing process.

In thicker liquid carriers (IDDSI L3 and L4), capsules and tablets were either pushed in front of the bolus or transported in the middle (TUC L3+capsule, and glycerol samples) (FIGS. 6 and 10). Therefore, all the liquid carriers tested improved the transport of the SODF considered in this study, except TUC L1, although this fluid led to low post swallow residues Indeed, the other criteria commented before (bolus shape, post-swallow residues, and oral transit times) should also be taken into account to decide which carrier to prefer.

Beta-glucan samples L1 and L3 both appear as very good options because they transported capsules and tablets at the front of a compact bolus, and only slightly increase oral transit times, without increasing too much the post-swallow residues.

### 1.3.9. Capsules Vs Tablets

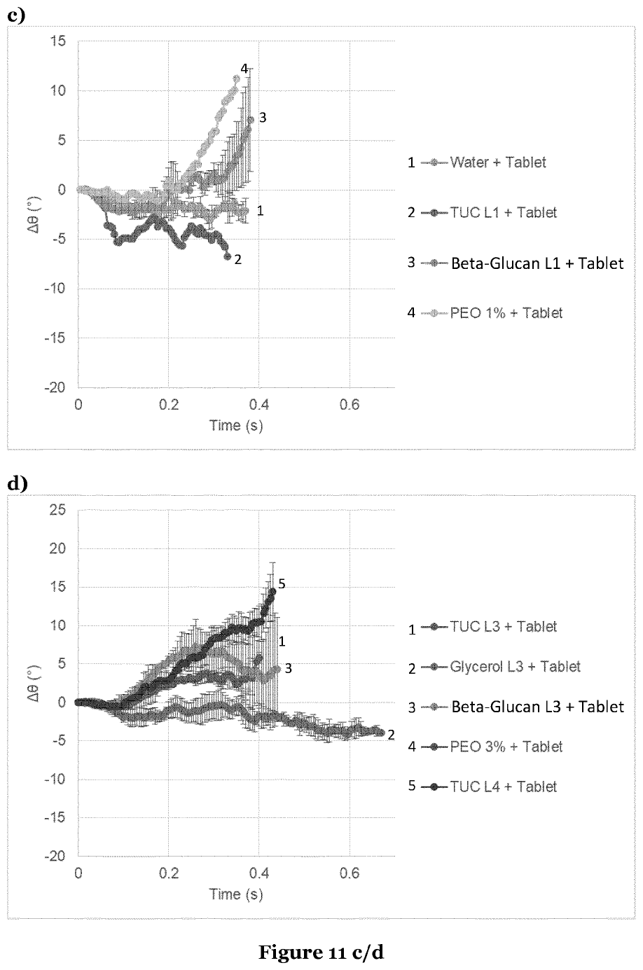

In order to further explore the differences between the transport of capsules and tablets during in vitro swallowing, the position of the SODF was also followed during the whole experiment. Data are presented in FIG. 11, separated by type of SODF and IDDSI levels.

Capsules seemed to adhere to the membrane mimicking the oral cavity during the first part of the experiment (i.e., t<0.15 s) with all the liquid carriers, except glycerol solutions (FIG. 11). At the beginning of the test, the capsule did not move while the liquid was able to flow forward (Δθ decreased). The capsule then reached the bolus tail (Δθ approx. −15°), and under the squeezing action imposed by the roller it finally detached from the sidewall. Then, during the last part of the experiment, two different scenarios were observed for the capsules. With water and TUC (L1 & L3), the capsule was pushed forward together with the liquid bolus (constant Δθ), while with the other carriers, the capsule moved faster than the liquid bolus (increasing Δθ) (FIG. 11).

When swallowed with glycerol (L1 & L3), no adhesion was observed between the capsules and the membrane, Δθ decreased continuously (FIG. 11) Tablets adhered significantly less to the membrane than the capsules at the beginning of the experiment (FIG. 11). With water, and glycerol (L3), Δθ decreased continuously during the experiment (FIG. 11). With the other liquid carriers, Δθ was first constant. Then, it increased around 0.1 s to reach the front of the bolus (Δθ approx. +15°), or a plateau around Δθ=5°, depending on the liquid carrier involved (FIG. 11). Overall, these results show that the tablets rapidly overcame the disadvantage of their initial position.

According to these results, the initial position of the SODF in the liquid bolus do not govern the subsequent evolution during swallowing. However, the adhesion of the SODF with the membrane had a significant impact on the swallowing dynamics of the solids and it should be further investigated.

Concerning the adhesion, one limitation of this study is that the contact time of the liquids and the SODF before triggering the in vitro swallowing was 2 min, which is longer than the typical in vivo contact time. Due to experimental constraints, it was not possible to reduce this immersion time.

In these experimental conditions, the uncoated tablet adhered less to the plastic membrane than the HPMC capsule. Since in glycerol solutions, neither the capsule nor the tablet seemed to adhere to the membrane, the differences observed could be due to a partial dissolution of the SODF surfaces in aqueous suspensions or to a lower adhesion in presence of glycerol solutions.

The adhesion of SODF to the mucus membranes from the oral cavity to the stomach has been investigated before, as it can be responsible of esophageal damage (Channer and Virjee 1986; Chisaka et al. 2006; Hey et al. 1982; Perkins et al. 1994). However, contradicting results can be found in the literature about the adhesion of HPMC capsules to the mucosa. On one hand, using an in vitro setup incorporating a section of porcine esophageal mucosa moistened with saliva Smart et al., (2013) concluded that tablets coated with HPMC had significant adhesive properties. On the other hand, static and kinetic friction coefficients between HPMC coated tablets and an artificial skin were shown to reduce almost to 0 when the capsules were previously immerged in water (Shimasaki et al. 2019). Authors considered that the HPMC coating acted as a lubricant between the formulation and the artificial skin, and concluded that this type of tablets would be easier to swallow than uncoated tablets when ingested with water.

### 2. Conclusions

These experiments used an in vitro artificial throat to study the dynamics of different sets of liquid carriers and SODF during the oral phase of swallowing. The effect of the rheological properties of the carriers on bolus velocity, bolus shape, post-swallow residues, and SODF position in the bolus were investigated. Experiments provided new insights on the transport of capsules and tablets in a peristaltic flow relevant to the oral phase of swallowing.

Low viscosity Newtonian fluids, like water, are not the most efficient carriers for SODF. When swallowed with water, capsules and tablets did not impact significantly the velocity of the bolus, but they lagged behind the liquid bolus, suggesting a higher risk of adhesion with the mucosa after the oral phase, because of the low kinetic energy of the liquid following the SODF.

The ability of the liquid to transport the SODF and their position in the bolus was improved by increasing the viscosity of the liquid carrier at high shear rates (i.e., ≥300 s−1). However, higher viscosities are associated with higher post-swallow residues, which could increase the risk of post-swallowing aspiration.

At equivalent shear viscosity, the position of the SODF in the bolus was positively affected by the elastic and extensional properties of the carriers. Capsules and tablets were transported toward the front of the bolus, which is considered more advantageous from a flow perspective, to maintain a drag on the SODF and prevent adhesion in the following phases of swallowing.

Thin elastic liquid formulations, like the beta-glucan sample evaluated in this study, therefore appear as an interesting option with a potential to promote swallowing of SODF. Clinical studies are however necessary to confirm if a positive effect is observed in dysphagic patients.

As a consequence, and surprising finding of the data, the inventors now found that a liquid viscoelastic swallowing aid for use in promoting swallowing of a Solid Oral Dosage Form (SODF) is most efficient, if the liquid viscoelastic swallowing aid comprises a total amount from 0.1 to 10 wt % of a compound selected from of beta-glucans or equivalent viscoelastic carriers selected from plant-derived mucilages and/or plant-extracted gums as defined herein. An optimum may be from 0.1 wt % to 5 wt %, from 0.1 wt % to 4.5 wt %, from 0.1 wt % to 3.5 wt %.

The liquid viscoelastic swallowing aid should preferably also exhibit:
a shear viscosity from 10 to 1,000 mPa·s, a shear viscosity from 10 to 900 mPa·s, a shear viscosity from 10 to 800 mPa·s, or a shear viscosity from 10-700 mPa·s measured at a shear rate of 50 s−1 and 25° C.; an optimum may be even lower, e.g., at 10-600 mPa·s, at 10-500 mPa·s, at 10-400 mPa·s, 10 to 300 mPa·s, at 10-200 mPa·s, e.g. around 10 to 100 mPa·s, 10 to 50 mPa·s, 10 to 40 mPa·s or even 10 to 30 mPa·s, each shear viscosity measured at a shear rate of 50 s−1 and 25° C.;
and at least one extensional relaxation time as measured by a Capillary Breakup Extensional Rheometer (CaBER) at room temperature (25° C.) from 10 to 500 ms or lower, e.g., down to 10 ms to 350 ms or even lower, and
optionally an IDDSI-level from 1 to 4, preferably an IDDSI-level of 1-3, more preferably an IDDSI-level of 1-2 or even 1, typically measured at room temperature (25° C.).

With such combined values, particularly in the scheduled optimum regions, inventors surprisingly found beneficial viscoelastic properties, such that a bolus containing an SODF experiences a moderate bolus extension, probably reducing fragmentation risks that may occur during swallowing; the SODF is swallowed in the middle or in front of the bolus such that no lagging behind of the SODF occurs, which is considered beneficial for the transport of the SODF.

### 3. References

References as cited hereinabove in brief are listed in the following in full-length as follows:
(1) Anna, Shelley L., and Gareth H. McKinley. 2000. ‘Elasto-Capillary Thinning and Breakup of Model Elastic Liquids’. Journal of Rheology 45(1):115-38. doi: 10.1122/1.1332389.
(2) Arnolds, Oliver, Hans Buggisch, Dirk Sachsenheimer, and Norbert Willenbacher. 2010. ‘Capillary Breakup Extensional Rheometry (CaBER) on Semi-Dilute and Concentrated Polyethyleneoxide (PEO) Solutions’. Rheologica Acta 49(11):1207-17. doi: 10.1007/s00397-010-0500-7.
(3) Channer, Kevin S., and James P. Virjee. 1986. ‘The Effect of Size and Shape of Tablets on Their Esophageal Transit’. The Journal of Clinical Pharmacology 26(2):141-46. doi: 10.1002/j.1552-4604.1986.tbo2922.x.
(4) Chisaka, Hiromi, Yasuyuki Matsushima, Futoshi Wada, Satoru Saeki, and Kenji Hachisuka. 2006. ‘Dynamics of Capsule Swallowing by Healthy Young Men and Capsule Transit Time from the Mouth to the Stomach’. Dysphagia 21(4):275-79. doi: 10.1007/s00455-006-9054-3.
(5) Diamond, Shelley, and Danielle C. Lavallee. 2010. ‘Experience With a Pill-Swallowing Enhancement Aid’. Clinical Pediatrics 49(4):391-93. doi: 10.1177/0009922809355313.
(6) Fields, Jeremy, Jorge T. Go, and Konrad S. Schulze. 2015. ‘Pill Properties That Cause Dysphagia and Treatment Failure’. Current Therapeutic Research, Clinical and Experimental 77:79-82. doi: 10.1016/j.curtheres.2015.08.002.
(7) Forough, Aida Sefidani, Esther T L Lau, Kathryn J. Steadman, Julie A Y Cichero, Greg J. Kyle, Jose Manuel Serrano Santos, and Lisa M. Nissen. 2018. ‘A Spoonful of Sugar Helps the Medicine Go down? A Review of Strategies for Making Pills Easier to Swallow’. Patient Preference and Adherence 12:1337-46. doi: 10.2147/PPA.S164406.
(8) Fukui, Atsuko. 2004. “‘Swallowing Aid Jelly” for Taking Medicines’. Journal of Pharmaceutical Science and Technology, Japan 64(4):240-44. doi: 10.14843/jpstj.64.240.
(9) Fukui, Atsuko. 2015. ‘Development of “Swallowing Aid Jelly” for Children’. Journal of Pharmaceutical Science and Technology, Japan 75(1):42-47. doi: 10.14843/jpstj.75.42.
(10) Gallegos, Críspulo, Lida Quinchia, Gabriel Ascanio, and Martín Salinas-Vázquez. 2012. ‘Rheology and Dysphagia: An Overview’. Annual Transactions of the Nordic Rheology Society 20:8.
(11) Hadde, Enrico Karsten, Wei Chen, and Jianshe Chen. 2020. ‘Cohesiveness Visual Evaluation of Thickened Fluids’. Food Hydrocolloids 101:105522. doi: 10.016/j.foodhyd.2019.105522.
(12) Hadde, Enrico Karsten, Julie Ann Yvette Cichero, Shaofeng Zhao, Wei Chen, and Jianshe Chen. 2019. ‘The Importance of Extensional Rheology in Bolus Control during Swallowing’. Scientific Reports 9(1):1-10. doi: 10.1038/s41598-019-52269-4.
(13) Heppner, Sieber, Esslinger, and Trögner. 2006. ‘Arzneimittelformen und Arzneimittelverabreichung in der Geriatrie’. Therapeutische Umschau 63(6):419-22. doi: 10.1024/0040-5930.63.6.419.
(14) Hey, H., F. Jorgensen, K. Sorensen, H. Hasselbalch, and T. Wamberg. 1982. ‘Oesophageal Transit of Six Commonly Used Tablets and Capsules.’ Br Med J(Clin Res Ed) 285(6356):1717-19. doi: 10.1136/bmj.285.6356.1717.
(15) Hoag, S. W. 2017. ‘Capsules Dosage Form’. Pp. 723-47 in Developing Solid Oral Dosage Forms. Elsevier.
(16) IDDSI. 2019. Complete IDDSI Framework Detailed Definitions 2.0.
(17) Jacobsen, Laura, Kathy Riley, Brian Lee, Kathleen Bradford, and Ravi Jhaveri. 2016. ‘Tablet/Capsule Size Variation Among the Most Commonly Prescribed Medications for Children in the USA: Retrospective Review and Firsthand Pharmacy Audit’. Paediatric Drugs 18(1):65-73. doi: 10.1007/s40272-015-0156-y.
(18) Kasashi, Kumiko, Kanchu Tei, Yasunori Totsuka, Takehiro Yamada, and Ken Iseki. 2011. ‘The Influence of Size, Specific Gravity, and Head Position on the Swallowing of Solid Preparations’. Oral Science International 8(2):55-59. doi: 10.1016/S1348-8643(11)00028-0.
(19) Lau, Esther T. L., Kathryn J. Steadman, Marilyn Mak, Julie A. Y. Cichero, and Lisa M. Nissen. 2015. ‘Prevalence of Swallowing Difficulties and Medication Modification in Customers of Community Pharmacists’. Journal of Pharmacy Practice and Research 45(1):18-23. doi: https://doi.org/10.1002/jppr.1052.
(20) Liu, Fang, Ambreen Ghaffur, Jackreet Bains, and Shaheen Hamdy. 2016. ‘Acceptability of Oral Solid Medicines in Older Adults with and without Dysphagia: A Nested Pilot Validation Questionnaire Based Observational Study’. International Journal of Pharmaceutics 512(2):374-81. doi: 10.1016/j.ijpharm.2016.03.007.
(21) Logrippo, Serena, Giovanna Ricci, Matteo Sestili, Marco Cespi, Letizia Ferrara, Giovanni F. Palmieri, Roberta Ganzetti, Giulia Bonacucina, and Paolo Blasi. 2017. ‘Oral Drug Therapy in Elderly with Dysphagia: Between a Rock and a Hard Place!’ Clinical Interventions in Aging 12:241-51. Retrieved 6 Apr. 2020 (https://www.dovepress.com/oral-drug-therapy-in-elderly-with-dysphagia-between-a-rock-and-a-hard-peer-reviewed-article-CIA).
(22) Mackley, M. R., C. Tock, R. Anthony, S. A. Butler, G. Chapman, and D. C. Vadillo. 2013. ‘The Rheology and Processing Behavior of Starch and Gum-Based Dysphagia Thickeners’. Journal of Rheology 57(6):1533-53. doi: 10.1122/1.4820494.
(23) Malouh, Marwa A., Julie A. Y. Cichero, Yady J. Manrique, Lucia Crino, Esther T. L. Lau, Lisa M. Nissen, and Kathryn J. Steadman. 2020. ‘Are Medication Swallowing Lubricants Suitable for Use in Dysphagia? Consistency, Viscosity, Texture, and Application of the International Dysphagia Diet Standardization Initiative (IDDSI) Framework’. Pharmaceutics 12(10):924. doi: 10.3390/pharmaceutics12100924.
(24) Marconati, M., S. Raut, A. Burbidge, J. Engmann, and M. Ramaioli. 2018. ‘An in Vitro Experiment to Simulate How Easy Tablets Are to Swallow’. International Journal of Pharmaceutics 535(1):27-37. doi: 10.1016/j.ijpharm.2017.10.028.
(25) Marconati, Marco, and Marco Ramaioli. 2020. ‘The Role of Extensional Rheology in the Oral Phase of Swallowing: An in Vitro Study’. ArXiv:2001.07810 [Cond-Mat, q-Bio].
(26) Masnoon, Nashwa, Sepehr Shakib, Lisa Kalisch-Ellett, and Gillian E. Caughey. 2017. ‘What Is Polypharmacy? A Systematic Review of Definitions’. BMC Geriatrics 17. doi: 10.1186/s12877-017-0621-2.
(27) McKinley, Gareth H. n.d. ‘VISCO-ELASTO-CAPILLARY THINNING AND BREAK-UP OF COMPLEX FLUIDS’. 50.
(28) Mowlavi, S., J. Engmann, A. Burbidge, R. Lloyd, P. Hayoun, B. Le Reverend, and M. Ramaioli. 2016. ‘In Vivo Observations and in Vitro Experiments on the Oral Phase of Swallowing of Newtonian and Shear-Thinning Liquids’. Journal of Biomechanics 49(16):3788-95. doi: 10.1016/j.jbiomech.2016.10.011.
(29) Newman, Roger, Natalia Vilardell, Pere Clavé, and Renee Speyer. 2016. ‘Effect of Bolus Viscosity on the Safety and Efficacy of Swallowing and the Kinematics of the Swallow Response in Patients with Oropharyngeal Dysphagia: White Paper by the European Society for Swallowing Disorders (ESSD)’. Dysphagia 31(2):232-49. doi: 10.1007/s00455-016-9696-8.
(30) Nishinari, Katsuyoshi, Makoto Takemasa, Tom Brenner, Lei Su, Yapeng Fang, Madoka Hirashima, Miki Yoshimura, Yoko Nitta, Hatsue Moritaka, Marta Tomczynska-Mleko, Stanislaw Mleko, and Yukihiro Michiwaki. 2016. ‘The Food Colloid Principle in the Design of Elderly Food: FOOD COLLOID PRINCIPLE’. Journal of Texture Studies 47(4):284-312. doi: 10.1111/jtxs.12201.
(31) Nishinari, Katsuyoshi, Mihaela Turcanu, Makoto Nakauma, and Yapeng Fang. 2019. ‘Role of Fluid Cohesiveness in Safe Swallowing’. Npj Science of Food 3(1):1-13. doi: 10.1038/s41538-019-0038-8.
(32) Nissen, Lisa M., Alison Haywood, and Kathryn J. Steadman. 2009. ‘Solid Medication Dosage Form Modification at the Bedside and in the Pharmacy of Queensland Hospitals’. Journal of Pharmacy Practice and Research 39(2):129-34. doi: https://doi.org/10.1002/j.2055-2335.2009.tb00436.x.
(33) Ortega, Omar, Mireia Bolívar-Prados, Viridiana Arreola, Weslania Viviane Nascimento, Noemí Tomsen, Crispulo Gallegos, Edmundo Brito-de La Fuente, and Pere Clavé. 2020. ‘Therapeutic Effect, Rheological Properties and α-Amylase Resistance of a New Mixed Starch and Xanthan Gum Thickener on Four Different Phenotypes of Patients with Oropharyngeal Dysphagia’. Nutrients 12(6):1873. doi: 10.3390/nu12061873.
(34) Overgaard, A. B. A., J. Møller-Sonnergaard, L. L. Christrup, J. Højsted, and R. Hansen. 2001. ‘Patients' Evaluation of Shape, Size and Colour of Solid Dosage Forms’. Pharmacy World and Science 23(5):185-88. doi: 10.1023/A:1012050931018.
(35) Patel, Simmi, Nathan Scott, Kavil Patel, Valentyn Mohylyuk, William J. McAuley, and Fang Liu. 2020. ‘Easy to Swallow “Instant” Jelly Formulations for Sustained Release Gliclazide Delivery’. Journal of Pharmaceutical Sciences. doi: 10.1016/j.xphs.2020.04.018.
(36) Perkins, A. C., C. G. Wilson, P. E. Blackshaw, R. M. Vincent, R. J. Dansereau, K. D. Juhlin, P. J. Bekker, and R. C. Spiller. 1994. ‘Impaired Oesophageal Transit of Capsule versus Tablet Formulations in the Elderly.’ Gut 35(10):1363-67. doi: 10.1136/gut.35.10.1363.
(37) Punzalan, Cecile, Daniel S. Budnitz, Stuart J. Chirtel, Andrew I. Geller, Olivia E. Jones, Robert P. Mozersky, and Beverly Wolpert. 2019. ‘Swallowing Problems and Dietary Supplements: Data From U.S. Food and Drug Administration Adverse Event Reports, 2006-2015’. Annals of Internal Medicine 171(10):771-73. doi: 10.7326/M19-0947.
(38) Radhakrishnan, Chandramouli. 2016. ‘Oral Medication Dose Form Alteration: Patient Factors and the Effect of Adding Thickened Fluids—UQ ESpace’. The University of Queensland, Australia.
(39) Rofes, L., V. Arreola, R. Mukherjee, J. Swanson, and P. Clavé. 2014. ‘The Effects of a Xanthan Gum-Based Thickener on the Swallowing Function of Patients with Dysphagia’. Alimentary Pharmacology & Therapeutics 39(10):1169-79. doi: 10.1111/apt.12696.
(40) Satyanarayana, Dixit A., Parthasarathi K. Kulkarni, and Hosakote G. Shivakumar. 2011. ‘Gels and Jellies as a Dosage Form for Dysphagia Patients: A Review’. Current Drug Therapy 6(2). doi: info:doi/10.2174/157488511795304921.
(41) Schiele, Julia T., Heike Penner, Hendrik Schneider, Renate Quinzler, Gabriele Reich,
Nikolai Wezler, William Micol, Peter Oster, and Walter E. Haefeli. 2015. ‘Swallowing Tablets and Capsules Increases the Risk of Penetration and Aspiration in Patients with Stroke-Induced Dysphagia’. Dysphagia 30(5):571-82. doi: 10.1007/s00455-015-9639-9.
(42) Schiele, Julia T., Renate Quinzler, Hans-Dieter Klimm, Markus G. Pruszydlo, and Walter E. Haefeli. 2013. ‘Difficulties Swallowing Solid Oral Dosage Forms in a General Practice Population: Prevalence, Causes, and Relationship to Dosage Forms’. European Journal of Clinical Pharmacology 69(4):937-48. doi: 10.1007/S00228-012-1417-0.
(43) Schiele, Julia T., Hendrik Schneider, Renate Quinzler, Gabriele Reich, and Walter E. Haefeli. 2014. ‘Two Techniques to Make Swallowing Pills Easier’. The Annals of Family Medicine 12(6):550-52. doi: 10.1370/afm.1693.
(44) Shaikh, Rahamatullah, Dónal P. O'Brien, Denise M. Croker, and Gavin M. Walker. 2018. ‘The Development of a Pharmaceutical Oral Solid Dosage Forms’. Pp. 27-65 in Computer Aided Chemical Engineering. Vol. 41. Elsevier.
(45) Shariff, Zakia B., Dania T. Dahmash, Daniel J. Kirby, Shahrzad Missaghi, Ali Rajabi-Siahboomi, and Ian D. Maidment. 2020. ‘Does the Formulation of Oral Solid Dosage Forms Affect Acceptance and Adherence in Older Patients? A Mixed Methods Systematic Review’. Journal of the American Medical Directors Association. doi: 10.1016/j.jamda.2020.01.108.
(46) Shimasaki, Maya, Nobuhiro Murayama, Yoshiaki Fujita, Akihiro Nakamura, and Tsutomu Harada. 2019. ‘A Novel Method to Quantitatively Evaluate Slipperiness and Frictional Forces of Solid Oral Dosage Forms and to Correlate These Parameters with Ease of Swallowing’. Journal of Drug Delivery Science and Technology 53:101141. doi: 10.1016/j.jddst.2019.101141.
(47) Smart, John D., Sian Dunkley, John Tsibouklis, and Simon Young. 2013. ‘An in Vitro Model for the Evaluation of the Adhesion of Solid Oral Dosage Forms to the Oesophagus’. International Journal of Pharmaceutics 447(1):199-203. doi: 10.1016/j.ijpharm.2013.02.017.
(48) Stegemann, S., M. Gosch, and J. Breitkreutz. 2012. ‘Swallowing Dysfunction and Dysphagia Is an Unrecognized Challenge for Oral Drug Therapy’. International Journal of Pharmaceutics 430(1):197-206. doi: 10.1016/j.ijpharm.2012.04.022.
(49) Strachan, I., and M. Greener. 2005. ‘Medication-Related Swallowing Difficulties May Be More Common than We Realise’. Pharmacy in Practice 15:411-14.
(50) Sukkar, Samir G., Norbert Maggi, Beatrice Travalca Cupillo, and Carmelina Ruggiero. 2018. ‘Optimizing Texture Modified Foods for Oro-Pharyngeal Dysphagia: A Difficult but Possible Target?’ Frontiers in Nutrition 5. doi: 10.3389/fnut.2018.00068.
(51) Sura, Livia, Aarthi Madhavan, Giselle Carnaby, and Michael A. Crary. 2012. ‘Dysphagia in the Elderly: Management and Nutritional Considerations’. Clinical Interventions in Aging 7:287-98. doi: 10.2147/CIA.S23404.
(52) Tahaineh, Linda, and Mayyada Wazaify. 2017. ‘Difficulties in Swallowing Oral Medications in Jordan’. International Journal of Clinical Pharmacy 39(2):373-79. doi: 10.1007/s11096-O17-0449-z.
(53) Uloza, Virgilijus, Ingrida Uloziene, and Egle Gradauskiene. 2010. ‘A Randomized Cross-over Study to Evaluate the Swallow-Enhancing and Taste-Masking Properties of a Novel Coating for Oral Tablets’. Pharmacy World & Science 32(4):420-23. doi: 10.1007/s11096-010-9399-4.
(54) United Nations. 2020. Word Population Ageing 2020 Highlights. Department of Economic and Social Affairs.
(55) U.S. Department of Health and Human Services Food and Drug Administration. 2013. Size, Shape, and Other Physical Attributes of Generic Tablets and Capsules.
(56) Wright, David John, John F. Potter, Allan Clark, Annie Blyth, Vivienne Maskrey, Giovanna Mencarelli, Sarah O. Wicks, and Duncan Q. M. Craig. 2019. ‘Administration of Aspirin Tablets Using a Novel Gel-Based Swallowing Aid: An Open-Label Randomised Controlled Cross-over Trial’. BMJ Innovations 5(4). doi: 10.1136/bmjinnov-2018-000293.
(57) Yamamoto, Shinya, Hiroshige Taniguchi, Hirokazu Hayashi, Kazuhiro Hori, Takanori Tsujimura, Yuki Nakamura, Hideaki Sato, and Makoto Inoue. 2014. ‘How Do Tablet Properties Influence Swallowing Behaviours?’ Journal of Pharmacy and Pharmacology 66(1):32-39. doi: 10.1111/jphp.12155.

1. A method for promoting safe swallowing of a Solid Oral Dosage Form (SODF) in a patient in need thereof, comprising a liquid viscoelastic swallowing aid, the liquid viscoelastic swallowing aid comprising a total amount from 0.1 to 10 wt % of a compound selected from beta-glucans, a plant-derived mucilage and/or a plant-extracted gum or a combination thereof, wherein the liquid viscoelastic swallowing aid comprises:
a shear viscosity from 10-1,000 mPa·s measured at a shear rate of 50 s−1 and 25° C.; and
at least one extensional relaxation time as measured by a Capillary Breakup Extensional Rheometer (CaBER) at room temperature (25° C.) from 10 to 1,000 ms.

2. The method according to claim 1, wherein the aid comprises an IDDSI level from 1 to 4.

3. The method according to claim 1, wherein the aid comprises a shear viscosity from 10 to 900 mPa·s, measured at a shear rate of 50 s−1 and 25° C.

4. The method according to claim 1, wherein the aid comprises an extensional relaxation time as measured by a Capillary Breakup Extensional Rheometer (CaBER) at room temperature (25° C.) from 10 to 900 ms.

5. The method according to claim 1, wherein a filament diameter of the liquid viscoelastic swallowing aid as measured by a Capillary Breakup Extensional Rheometer (CaBER) at room temperature (25° C.) decreases exponentially in time during the CaBER experiment.

6. The method according to claim 1, wherein the plant-extracted gum is selected from the group consisting of okra gum, konjac mannan, tara gum, locust bean gum, guar gum, fenugreek gum, tamarind gum, cassia gum, acacia gum, gum ghatti, pectins, cellulosics, tragacanth gum, karaya gum, and any combinations thereof.

7. The method according to claim 1, wherein the plant-derived mucilage is selected from the group consisting of cactus mucilage, psyllium mucilage, mallow mucilage, flax seed mucilage, marshmallow mucilage, ribwort mucilage, mullein mucilage, cetraria mucilage, and combinations thereof.

8. The method according to claim 1, wherein the beta-glucans, plant-derived mucilage and/or plant-extracted gum is present in a total amount from 0.01 wt % to 10 wt %.

9. The method according to claim 1, wherein the Solid Oral Dosage Form (SODF) is a tablet or a capsule.

10. The method according to claim 1, wherein the patient is suffering from a swallowing disorder.

11. The method according to claim 1, wherein the liquid viscoelastic swallowing aid is in an administrable form selected from the group consisting of pharmaceutical formulations, dietary supplements, functional beverage products, food for special medical purpose (FSMP), and combinations thereof.

12. The method according to claim 1, wherein the liquid viscoelastic swallowing aid is provided in a concentrated form to be diluted prior to use or is provided in a ready-to-use form, or is provided as a powder to be reconstituted prior to use.

13. The method according to claim 1, wherein the liquid viscoelastic swallowing aid furthermore contains an eptienal-ingredient selected from the group consisting of food additives, acidulants, buffers or agents for pH adjustment, chelating agents, colorants, emulsifiers, excipient, flavour agent, minerals, osmotic agents, a pharmaceutically acceptable carrier, preservatives, stabilisers, sugar(s), sweetener(s), texturiser(s), vitamin(s), proteins, lipids and carbohydrates.

14. A liquid viscoelastic swallowing aid for promoting safe swallowing of a Solid Oral Dosage Form (SODF) in a healthy person, the liquid viscoelastic swallowing aid comprising a total amount from 0.1 to 10 wt % of a compound selected from the group consisting of beta-glucans, a plant-derived mucilage, a plant-extracted gum and a combination thereof, wherein the liquid viscoelastic swallowing aid comprises:
a shear viscosity from 10-1,000 mPa·s measured at a shear rate of 50 s−1 and 25° C.; and
at least one extensional relaxation time as measured by a Capillary Breakup Extensional Rheometer (CaBER) at room temperature (25° C.) from 10 to 1,000 ms; and
an IDDSI-level from 1 to 4 measured at room temperature (25° C.).

15. The swallowing aid according to claim 14, wherein the composition comprises
an IDDSI level from 1 to 3;
a shear viscosity from 10 to 900 mPa·s; and
an extensional relaxation time as measured by a Capillary Breakup Extensional Rheometer (CaBER) at room temperature (25° C.) from 10 to 900 ms.

16. The swallowing aid according to claim 14, wherein a filament diameter of the liquid viscoelastic swallowing aid as measured by a Capillary Breakup Extensional Rheometer (CaBER) at room temperature (25° C.) decreases exponentially in time during the CaBER experiment.

17. The swallowing aid according to claim 14, wherein
the plant-extracted gum is selected from the group consisting of okra gum, konjac mannan, tara gum, locust bean gum, guar gum, fenugreek gum, tamarind gum, cassia gum, acacia gum, gum ghatti, pectins, cellulosics, tragacanth gum, karaya gum, and any combinations thereof; and/or
the plant-derived mucilages is selected from the group consisting of cactus mucilage, psyllium mucilage, mallow mucilage, flax seed mucilage, marshmallow mucilage, ribwort mucilage, mullein mucilage, cetraria mucilage, and combinations thereof.

18. The swallowing aid according to claim 14, wherein, wherein the beta-glucans, plant-derived mucilage and/or plant-extracted gum is present in a total amount from 0.01 wt % to 10 wt %.

19. The swallowing aid according to claim 14, wherein the Solid Oral Dosage Form (SODF) is a tablet or a capsule.
